(19) United States
(12) Patent Application Publication
(10) Pub. No.: US 2012/0178698 A1
(43) Pub. Date: Jul. 12, 2012

(54) Agents and Uses Thereof

(76) Inventors: Carl Arne Krister Borrebaeck, Lund (SE); Sara Charlotte Andersson Ek, Lund (SE)

(21) Appl. No.: 13/378,805

(22) PCT Filed: Jun. 18, 2010
(86) PCT No.: PCT/GB2010/001200

§ 371 Date: Feb. 24, 2012

(30) Foreign Application Priority Data
Jun. 19, 2009 (GB) 0910620.4

(51) Int. Cl. A61K 38/17; A61P 35/00; A61K 48/00

(52) U.S. Cl. 514/19.3; 514/44 R

(57) ABSTRACT

The present invention provides agents capable of activating Sox11 for use in medicine. In particular, the agents of the invention are useful in the treatment of cancers, such as lymphomas (e.g. mantel cell lymphoma). The invention further provides pharmaceutical compositions of the agents of the invention, as well as methods and uses of the same.

### FIELD OF THE INVENTION

The present invention provides agents capable of activating Sox11 for use in medicine. In particular there are provided agents and pharmaceutical compositions thereof capable of modulating the activity of Sox11 for use in the treatment of cancers (such as lymphomas).

### BACKGROUND

The neural transcription factor Sox11 is a diagnostic antigen for mantle cell lymphoma (MCL)¹ and nuclear expression of Sox11 has recently been claimed to be indicative of prolonged overall survival in MCL.² Recent investigations demonstrated that nuclear expression of Sox11 is also observed in Burkitt Lymphoma (BL) and precursor B and T cell lymphoblastic neoplasia³, indicating a more widespread presence in lymphoproliferative disease cells than initially anticipated. Furthermore, analysis of solid tumors revealed a strong nuclear expression of Sox11 in epithelial ovarian cancer (EOC), which was shown to correlate to a prolonged recurrence-free survive. It is previously known that Sox11 is highly abundant in both the fetal central nervous system (CNS) and CNS derived malignancies such as medulloblastoma⁵ and malignant glioma.⁶

To date, the main role of Sox11 in non-malignant tissues has been its necessity for neural development^{8,9} and organogenesis¹⁰ during fetal development, although the regulatory mechanisms remain unclear. Sox11 belongs to a group of 20 transcription factors within the high-mobility group (HMG) box protein super family, which are characterized by high sequence homology within their DNA-binding HMG domain.¹¹ A large variability exists outside this domain enabling Sox proteins to partner with different proteins¹². In vitro data have shown that Sox11 partners with Oct-3 and Brn-2 leading to activation of transcription.¹³ Others have shown that the interaction between Sox11 and Brn-1 was dependent on binding of both proteins to adjacent DNA elements and required the presence of their respective transactivation domains¹⁰ Thus, there is growing support for a model in which the HMG domain serves two functions, i.e. DNA binding as well as partner selection, which may permit a selective recruitment of Sox proteins to specific genes and transcription factors.

Despite extensive study, a therapeutic value of Sox11 in human disease has yet to be identified. As detailed above, Sox11 has been suggested primarily for use in diagnostic methods.

Inevitably, there remains an ongoing need for new therapies for the treatment of human diseases. Thus, the present invention seeks to provide new therapeutic agents for the treatment of cancer.

### SUMMARY OF THE INVENTION

A first aspect of the invention provides an agent capable of activating Sox11 for use in medicine. For the avoidance of doubt, the first aspect of the invention and all of its embodiments (stipulated below), also include and/or relate to the use of an agent capable of activating Sox11 in the preparation of a medicament for use in medicine.

By an “agent” we include all chemical entities, for example oligonucleotides, polynucleotides, polypeptides, peptidomimetics and small compounds.

By “activating Sox11” we specifically include the ability to increase:
(a) the amount or stability of Sox11 mRNA;
(b) the amount or stability of Sox11 protein;
(c) the binding of Sox11 to and/or activation of its cognate receptor(s);
(d) the binding of Sox11 to and/or activation of its binding partners (including Oct-3, Brn-1 and Brn-2); and
(e) Sox11-associated downstream signalling.

Thus, the agents of the invention may be any moiety which increases Sox11-mediated signalling events within the cell, either by an indirect or direct action upon Sox11 protein or by modulation of upstream or downstream signalling effector molecules.

Such agents may be identified using methods well known in the art, for example:
(i) by determining the effect of a test agent on levels of expression of Sox11 mRNA, for example by Southern blotting or related hybridisation techniques;
(ii) by determining the effect of a test agent on levels of Sox11 protein, for example by immunoassays using anti-Sox11 antibodies; and
(iii) by determining the effect of a test agent on inhibition in vitro or in vivo of cancer cell proliferation, for example by Methyl-3H-Thymidine (MTT) incorporation (see Example A).

Advantageously, the agent is capable of activating Sox11 selectively.

By ‘selectively’ we mean that the agent activates Sox11 to a greater extent than it activates other proteins. Preferably, the agent only activates Sox11, although it will be appreciated that the expression and activity of other proteins within the cancer cells may change as a downstream consequence of activating Sox11. Thus, we exclude agents which have a substantially non-specific effect on gene expression and/or cancer cell growth.

A second aspect of the invention provides an agent capable of activating Sox11 for use in the treatment of cancer. For the avoidance of doubt, the second aspect of the invention and all of its embodiments (stipulated below), also include and/or relate to the use of an agent capable of activating Sox11 in the preparation of a medicament for use in the treatment of cancer.

In one embodiment, the cancer is selected from the group consisting of cancers of the breast, bile duct, central nervous system (e.g. brain) and other nerve cells, colon, stomach, reproductive organs, lung and airways, skin, gallbladder, liver, nasopharynx, kidney, prostate, lymph glands, bones (including bone marrow), spleen, blood and gastrointestinal tract.

In a further embodiment, the cancer is a lymphoma or leukaemia.

Thus, the lymphoma or leukaemia may be selected from the group of lymphomas and leukaemias listed in Table 1.

TABLE 1
WHO classification of the mature B-cell, T-cell, and
NK-cell neoplasms (2008)
Mature B-cell neoplasms
Chronic lymphocytic leukemia/small lymphocytic lymphoma
B-cell prolymphocytic leukemia
Splenic marginal zone lymphoma
Hairy cell leukemia
Splenic lymphoma/leukemia, unclassifiable*
Splenic diffuse red pulp small B-cell lymphoma*
Hairy cell leukemia-variant*
Lymphoplasmacytic lymphoma
Waldenström macroglobulinemia
Heavy chain diseases
Alpha heavy chain disease
Gamma heavy chain disease
Mu heavy chain disease
Plasma cell myeloma
Solitary plasmacytoma of bone
Extraosseous plasmacytoma
Extranodal marginal zone lymphoma of mucosa-associated
lymphoid tissue
(MALT lymphoma)
Nodal marginal zone lymphoma
Pediatric nodal marginal zone lymphoma*
Follicular lymphoma
Pediatric follicular lymphoma*
Primary cutaneous follicle center lymphoma
Mantle cell lymphoma
Diffuse large B-cell lymphoma (DLBCL), NOS
T-cell/histiocyte rich large B-cell lymphoma
Primary DLBCL of the CNS
Primary cutaneous DLBCL, leg type
EBV⁺ DLBCL of the elderly*
DLBCL associated with chronic inflammation
Lymphomatoid granulomatosis
Primary mediastinal (thymic) large B-cell lymphoma
Intravascular large B-cell lymphoma
ALK⁺ large B-cell lymphoma
Plasmablastic lymphoma
Large B-cell lymphoma arising in HHV8-associated multicentric
Castleman disease
Primary effusion lymphoma
Burkitt lymphoma
B-cell lymphoma, unclassifiable, with features intermediate between
diffuse large B-cell lymphoma and Burkitt lymphoma
B-cell lymphoma, unclassifiable, with features intermediate between
diffuse large B-cell lymphoma and classical Hodgkin lymphoma
Mature T-cell and NK-cell neoplasms
T-cell prolymphocytic leukemia
T-cell large granular lymphocytic leukemia
Chronic lymphoproliferative disorder of NK cells*
Aggressive NK cell leukemia
Systemic EBV⁺ T-cell lymphoproliferative disease of childhood
Hydroa vacciniforme-like lymphoma
Adult T-cell leukemia/lymphoma
Extranodal NK/T-cell lymphoma, nasal type
Enteropathy-associated T-cell lymphoma
Hepatosplenic T-cell lymphoma
Subcutaneous panniculitis-like T-cell lymphoma
Mycosis fungoides
Sézary syndrome
Primary cutaneous CD30⁺ T-cell lymphoproliferative disorders
Lymphomatoid papulosis
Primary cutaneous anaplastic large cell lymphoma
Primary cutaneous gamma-delta T-cell lymphoma
Primary cutaneous CD8⁺ aggressive epidermotropic cytotoxic
T-cell lymphoma*
Primary cutaneous CD4⁺ small/medium T-cell lymphoma*
Peripheral T-cell lymphoma, NOS
Angioimmunoblastic T-cell lymphoma
Anaplastic large cell lymphoma, ALK⁺
Anaplastic large cell lymphoma, ALK⁻*
Hodgkin lymphoma
Nodular lymphocyte-predominant Hodgkin lymphoma
Classical Hodgkin lymphoma
Nodular sclerosis classical Hodgkin lymphoma
Lymphocyte-rich classical Hodgkin lymphoma
Mixed cellularity classical Hodgkin lymphoma
Lymphocyte-depleted classical Hodgkin lymphoma
Posttransplantation lymphoproliferative disorders (PTLD)
Early lesions
Plasmacytic hyperplasia
Infectious mononucleosis-like PTLD
Polymorphic PTLD
Monomorphic PTLD (B- and T/NK-cell types)^{†}
Classical Hodgkin lymphoma type PTLD^{†}
*Provisional entities for which the WHO Working Group felt there was insufficient evidence to recognize as distinct diseases at this time.
^{†}These lesions are classified according to the leukemia or lymphoma to which they correspond.

Thus, the lymphoma or leukaemia may be a B cell lymphoma.

For example, the lymphoma may be a follicular lymphoma (FL), a mantle cell lymphoma (MCL) or a diffuse large B cell lymphoma (DLBCL).

In an alternative embodiment, the cancer is an acute monocytic leukaemia.

For example, the acute monocytic leukaemia may be an acute myeloid leukemia (AML).

In a further alternative embodiment, the cancer is a cancer of epithelial cells. For example, the cancer may be epithelial ovarian cancer (EOC).

In one preferred embodiment, the agent is capable of inhibiting the proliferation of cancer cells.

The cancer cells may be Sox11-expressing (for example, MCL or DLBCL) or non-Sox11-expressing (for example, FL).

Advantageously, the agent is capable of inhibiting the proliferation of cancer cells in vivo.

In one embodiment, the agent is capable of inhibiting the proliferation of cancer cells by 20% or more compared to the proliferation of cancer cells which have not been exposed to the agent, for example by at least 30%, 40%, 50%, 60%, 70%, 80%, 90% or more.

In another preferred embodiment, the agent is capable of increasing the rate of cancer cell death.

Advantageously, the agent is capable of inhibiting the proliferation of cancer cells in vivo.

In one embodiment the agent is capable of increasing the rate of cancer cell death by 20% or more compared to the rate of cell death of cancer cells which have not been exposed to the agent, for example by at least 30%, 40%, 50%, 60%, 70%, 80%, 90% or more.

As detailed above, the agents for use in the invention may activate Sox11 by any suitable means. For example, the agent may increase the transcription, translation, binding properties, biological activity and/or stability of Sox11, and/or signalling induced thereby.

In one embodiment, the agent increases the transcription of Sox11. For example, the agent may reduce, prevent or inhibit the methylation of the Sox11 promoter region. Alternatively, the agent may increase the stability of the Sox11 transcript (i.e. Sox11 mRNA).

In a further embodiment, the agent increases the translation of Sox11.

In a still further embodiment, the agent increases the binding properties of Sox11. For example, the agent may increase the binding of Sox11 to, and/or activation of, its binding partners, such as Oct-3, Brn-1 and/or Brn-2.

Methods for detecting interactions between a test compound and target proteins are well known in the art. For example ultrafiltration with ion spray mass spectroscopy/HPLC methods or other physical and analytical methods may be used. In addition, Fluorescence Energy Resonance Transfer (FRET) methods may be used, in which binding of two fluorescent labelled entities may be measured by measuring the interaction of the fluorescent labels when in close proximity to each other.

Alternative methods of detecting binding of a polypeptide to macromolecules, for example DNA, RNA, proteins and phospholipids, include a surface plasmon resonance assay, for example as described in Plant et al., 1995, Analyt Biochem 226(2), 342-348. Methods may make use of a polypeptide that is labelled, for example with a radioactive or fluorescent label.

In a further embodiment, the agent increases the biological activity of (endogenous) Sox11 protein.

In another embodiment, the agent increases the stability of Sox11 (either at the mRNA or protein level).

In a still further embodiment, the agent increases Sox11-mediated signalling.

It will be appreciated by persons skilled in the art that an increase in Sox11-mediated signalling may be achieved through a direct effect (e.g. on Sox11 mRNA and/or protein) and/or through an indirect effect (e.g. on the upstream and/or downstream signalling effectors).

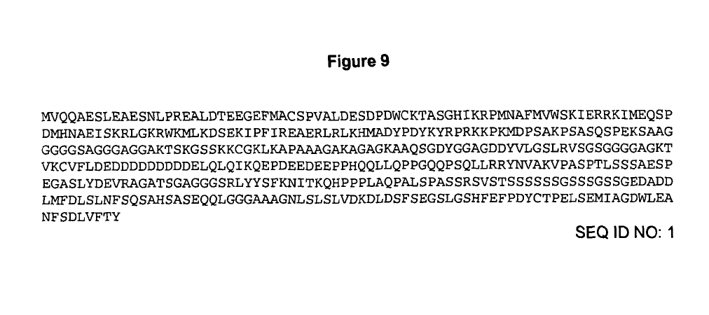

Thus, in one embodiment, the agent comprises or consists of a polypeptide according to SEQ ID NO: 1 (see FIG. 9) or a biologically active fragment, variant, fusion or derivative thereof.

SEQ ID NO: 1 corresponds to the human Sox11 protein (see also Database Accession Nos. BAA88122, AAH25789, and AAB08518).

The term “polypeptide” as used herein takes its conventional meaning unless otherwise specified, namely a plurality of amino acids that are linked together via a peptide bond.

In the formulas representing polypeptide embodiments of the present invention, the amino- and carboxy-terminal groups, although often not specifically shown, will be understood to be in the form they would assume at physiological pH values, unless otherwise specified. Thus, the N-terminal H²⁺ and C-terminal O⁻ at physiological pH are understood to be present though not necessarily specified and shown, either in specific examples or in generic formulas. In the polypeptide notation used herein, the left-hand end of the molecule is the amino terminal end and the right-hand end is the carboxy-terminal end, in accordance with standard usage and convention. The basic and acid addition salts including those which are formed at non-physiological pH values are also included in the polypeptides of the invention.

The term ‘amino acid’ as used herein includes the standard twenty genetically-encoded amino acids and their corresponding stereoisomers in the ‘D’ form (as compared to the natural ‘L’ form), omega-amino acids other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

For example, the polypeptides of the invention may comprise or consist of L-amino acids.

In one preferred embodiment, the agent comprises or consists of a polypeptide according to SEQ ID NO: 1.

In an alternative preferred embodiment, the agent comprises or consists of a biologically active fragment of a polypeptide according to SEQ ID NO: 1. Thus, the fragment may comprise or consist of at least 100 contiguous amino acid of SEQ ID NO: 1, for example at least 5, 10, 15, 25, 35, 50, 75, 100, 125, 150, 200, 250, 300, 350, 400 or 440 contiguous amino acids of SEQ ID NO: 1.

By “biologically active fragment” it is meant a fragment of Sox11 that retains an activity of the wild type Sox11 polypeptide. In particular, the fragment retains the ability of the parent Sox11 protein to inhibit the proliferation of cancer cells.

In another embodiment, the agent comprises or consists of a biologically active variant of a polypeptide according to SEQ ID NO: 1, or fragment thereof. Thus, the variant may share at least 70% sequence identity with a polypeptide according to SEQ ID NO: 1, or fragment thereof, for example at least 80%, 85%, 90%, 95%, 96%, 97%, 98% or 99% sequence identity.

By “biologically active variant” it is meant a variant of Sox11 that retains an activity of the wild type Sox11 polypeptide (see above).

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nuc. Acid Res. 22:4673-4680).

The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

Variants of a known amino acid sequence may be made using the methods well known in the art (for example, as described in Molecular Cloning: A Laboratory Manual, 3rd edition, Sambrook & Russell, 2001, Cold Spring Harbor Laboratory Press, the relevant disclosures in which document are hereby incorporated by reference). For example, sequence variation may be introduced using error prone PCR (Leung et al., Technique, 1: 11-15, 1989), the GeneMorph II™ random mutagenesis kit (Stratagene) and other known methods of random mutagenesis, site-directed mutagenesis and protein engineering.

Persons skilled in the art will appreciate that nucleic acid-based agents may also be used as activators of Sox11.

Thus, in an alternative embodiment, the agent comprises or consists of a nucleic acid molecule encoding a polypeptide according to SEQ ID NO: 1 or a biologically active fragment, variant, fusion or derivative thereof.

For example, the agent may comprise or consist of a nucleic acid molecule encoding a polypeptide according to SEQ ID NO: 1.

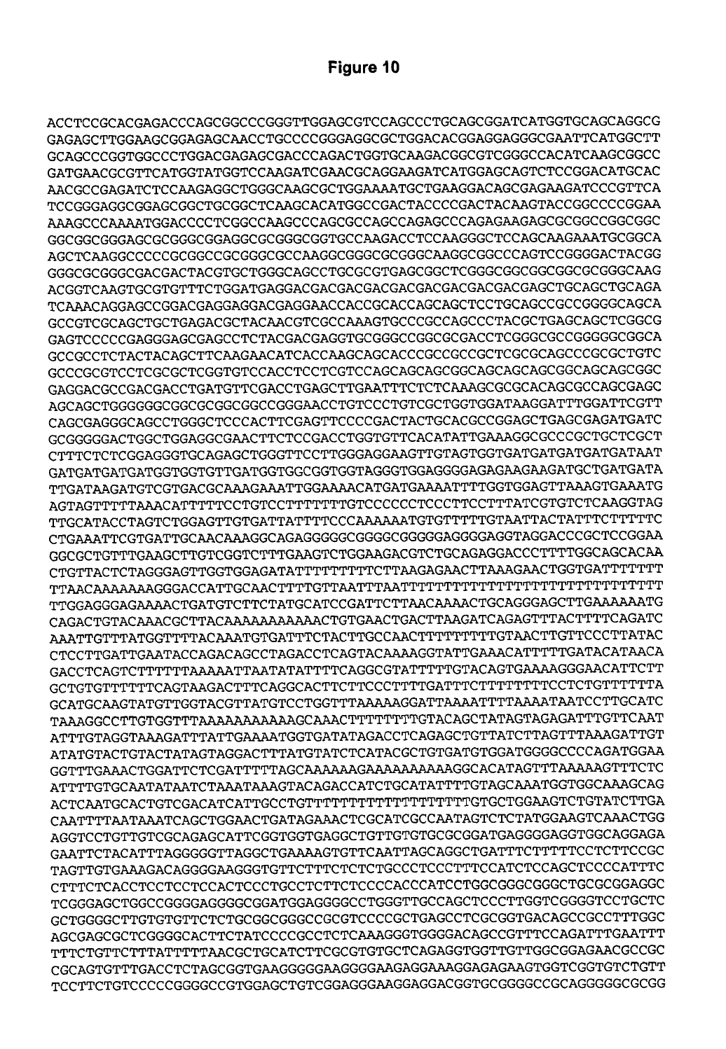

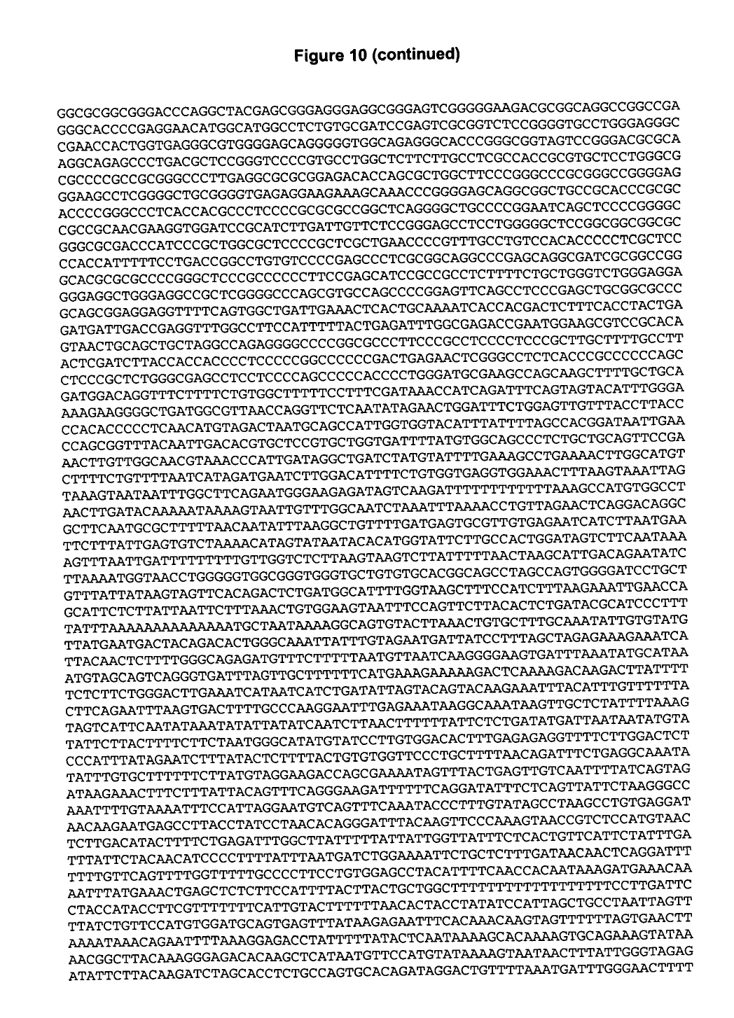

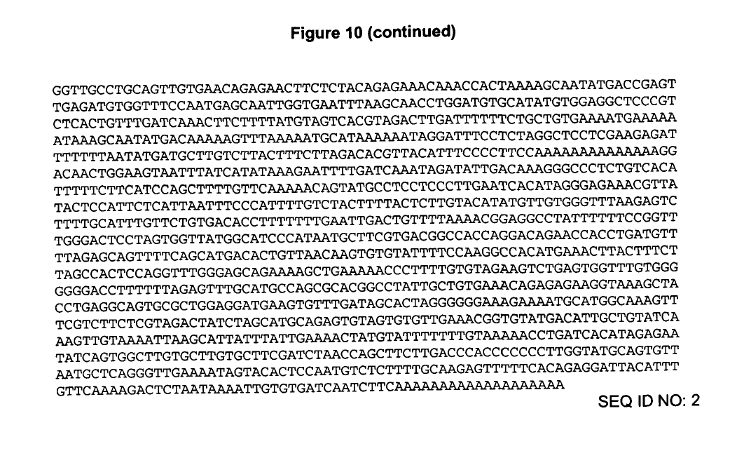

In one preferred embodiment, the nucleic acid molecule comprises or consists of a nucleotide sequence according to SEQ ID NO: 2 (see FIG. 10) or a fragment, variant, fusion or derivative thereof. Alternatively, the nucleic acid molecule may comprise or consist of a degenerate of such a nucleotide sequence.

Advantageously, the nucleic acid molecule comprises or consists of DNA, RNA, PNA (Peptide Nucleic Acid), LNA (Locked Nucleic Acid), GNA (Glycol Nucleic Acid), TNA (Threose Nucleic Acid) or PMO (Phosphorodiamidate Morpholino Oligomer). Preferably the nucleic acid molecule comprises or consists of cDNA or mRNA.

In one embodiment, the nucleic acid may comprise a sequence encoding nuclear location signal.

It will be further appreciated by person skilled in the art that oligonucleotides are subject to being degraded or inactivated by cellular endogenous nucleases. To counter this problem, it is possible to use modified oligonucleotides, e.g. having altered internucleotide linkages, in which the naturally occurring phosphodiester linkages have been replaced with another linkage. For example, Agrawal et al (1988) Proc. Natl. Acad, Sci. USA 85, 7079-7083 showed increased inhibition in tissue culture of HIV-1 using oligonucleotide phosphoramidates and phosphorothioates. Sarin et al (1988) Proc. Natl. Acad. Sci. USA 85, 7448-7451 demonstrated increased inhibition of HIV-1 using oligonucleotide methylphosphonates. Agrawal et al (1989) Proc. Natl. Acad. Sci. USA 86, 7790-7794 showed inhibition of HIV-1 replication in both early-infected and chronically infected cell cultures, using nucleotide sequence-specific oligonucleotide phosphorothioates. Leither et al (1990) Proc. Natl. Acad. Sci. USA 87, 3430-3434 report inhibition in tissue culture of influenza virus replication by oligonucleotide phosphorothioates.

Oligonucleotides having artificial linkages have been shown to be resistant to degradation in vivo. For example, Shaw et al (1991) in Nucleic Acids Res. 19, 747-750, report that otherwise unmodified oligonucleotides become more resistant to nucleases in vivo when they are blocked at the 3′ end by certain capping structures and that uncapped oligonucleotide phosphorothioates are not degraded in vivo.

A detailed description of the H-phosphonate approach to synthesising oligonucleoside phosphorothioates is provided in Agrawal and Tang (1990) Tetrahedron Letters 31, 7541-7544, the teachings of which are hereby incorporated herein by reference. Syntheses of oligonucleoside methylphosphonates, phosphorodithioates, phosphoramidates, phosphate esters, bridged phosphoramidates and bridge phosphorothioates are known in the art. See, for example, Agrawal and Goodchild (1987) Tetrahedron Letters 28, 3539; Nielsen et al (1988) Tetrahedron Letters 29, 2911; Jager et al (1988) Biochemistry 27, 7237; Uznanski et al (1987) Tetrahedron Letters 28, 3401; Bannwarth (1988) Helv. Chim. Acta. 71, 1517; Crosstick and Vyle (1989) Tetrahedron Letters 30, 4693; Agrawal et al (1990) Proc. Natl. Acad. Sci. USA 87, 1401-1405, the teachings of which are incorporated herein by reference. Other methods for synthesis or production also are possible. In a preferred embodiment the oligonucleotide is a deoxyribonucleic acid (DNA), although ribonucleic acid (RNA) sequences may also be synthesised and applied.

The oligonucleotides useful in the invention preferably are designed to resist degradation by endogenous nucleolytic enzymes. In vivo degradation of oligonucleotides produces oligonucleotide breakdown products of reduced length. Such breakdown products are more likely to engage in non-specific hybridisation and are less likely to be effective, relative to their full-length counterparts. Thus, it is desirable to use oligonucleotides that are resistant to degradation in the body and which are able to reach the targeted cells. The present oligonucleotides can be rendered more resistant to degradation in vivo by substituting one or more internal artificial internucleotide linkages for the native phosphodiester linkages, for example, by replacing phosphate with sulphur in the linkage. Examples of linkages that may be used include phosphorothioates, methylphosphonates, sulphone, sulphate, ketyl, phosphorodithioates, various phosphoramidates, phosphate esters, bridged phosphorothioates and bridged phosphoramidates. Such examples are illustrative, rather than limiting, since other internucleotide linkages are well known in the art. The synthesis of oligonucleotides having one or more of these linkages substituted for the phosphodiester internucleotide linkages is well known in the art, including synthetic pathways for producing oligonucleotides having mixed internucleotide linkages.

Oligonucleotides can be made resistant to extension by endogenous enzymes by “capping” or incorporating similar groups on the 5′ or 3′ terminal nucleotides. A reagent for capping is commercially available as Amino-Link II™ from Applied BioSystems Inc, Foster City, Calif. Methods for capping are described, for example, by Shaw et al (1991) Nucleic Acids Res. 19, 747-750 and Agrawal et al (1991) Proc. Natl. Acad. Sci. USA 88(17), 7595-7599.

A further method of making oligonucleotides resistant to nuclease attack is for them to be “self-stabilised” as described by Tang et al (1993) Nucl. Acids Res. 21, 2729-2735. Self-stabilised oligonucleotides have hairpin loop structures at their 3′ ends, and show increased resistance to degradation by snake venom phosphodiesterase, DNA polymerase I and foetal bovine serum. The self-stabilised region of the oligonucleotide does not interfere in hybridisation with complementary nucleic acids, and pharmacokinetic and stability studies in mice have shown increased in vivo persistence of self-stabilised oligonucleotides with respect to their linear counterparts.

In one embodiment, the agent comprises or consists of a gene therapy vector, such as a plasmid or a virus.

For example, the virus or plasmid may be selected from the group consisting of retrovirus, adenovirus, adeno-associated virus, herpes simplex virus 1 (HSV-1), lentiviruses, foamy virus based vectors and reovirus.

Methods for administering oligonucleotide or polynucleotide agents of the invention are also well known in the art (see Dass, 2002, J Pharm Pharmacol. 54(1):3-27; Dass, 2001, Drug Deliv. 8(4):191-213; Lebedeva et al., 2000, Eur J Pharm Biopharm. 50(1):101-19; Pierce et al., 2005, Mini Rev Med Chem. 5(1):41-55; Lysik & Wu-Pong, 2003, J Pharm Sci. 2003 2(8):1559-73; Dass, 2004, Biotechnol Appl Biochem. 40(Pt 2):113-22; Medina, 2004, Curr Pharm Des. 10(24):2981-9.

For example, the constructs of the invention may be introduced into cells by methods involving retroviruses, so that the construct is inserted into the genome of the cell. For example, in Kuriyama et al (1991) Cell Struc. and Func. 16, 503-510 purified retroviruses are administered. Retroviral DNA constructs comprising a polynucleotide as described above may be made using methods well known in the art. To produce active retrovirus from such a construct it is usual to use an ecotropic psi2 packaging cell line grown in Dulbecco's modified Eagle's medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neo^{R} gene). Independent colonies are isolated and expanded and the culture supernatant removed, filtered through a 0.45 μm pore-size filter and stored at −70° C. For the introduction of the retrovirus into the tumour cells, it is convenient to inject directly retroviral supernatant to which 10 μg/ml Polybrene has been added. For tumours exceeding 10 mm in diameter it is appropriate to inject between 0.1 ml and 1 ml of retroviral supernatant; preferably 0.5 ml.

Alternatively, as described in Culver et al (1992) Science 256, 1550-1552, cells which produce retroviruses are injected. The retrovirus-producing cells so introduced are engineered to actively produce retroviral vector particles so that continuous productions of the vector occurred within the tumour mass in situ. Thus, proliferating cells can be successfully transduced in vivo if mixed with retroviral vector-producing cells.

Targeted retroviruses are also available for use in the invention; for example, sequences conferring specific binding affinities may be engineered into pre-existing viral env genes (see Miller & Vile (1995) Faseb J. 9, 190-199 for a review of this and other targeted vectors for gene therapy).

Other methods involve simple delivery of the construct into the cell for expression therein either for a limited time or, following integration into the genome, for a longer time. An example of the latter approach includes liposomes (Nassander et al (1992) Cancer Res. 52, 646-653).

For the preparation of immuno-liposomes MPB-PE (N-[4-(p-maleimidophenyl)butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the agent of the invention (such as DNA or other genetic construct) for delivery to the target cells, for example, by forming the said liposomes in a solution of the agent, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80 000×g for 45 min. Immunoliposomes may be injected intraperitoneally or directly into the tumour.

Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with an oligonucleotide agent of the invention, wherein the antibody is specific for either wild-type adenovirus or a variant adenovirus in which a new epitope has been introduced which binds the antibody. The polycation moiety binds the oligonucleotide agent via electrostatic interactions with the phosphate backbone. The adenovirus, because it contains unaltered fibre and penton proteins, is internalised into the cell and carries into the cell with it the oligonucleotide agent of the invention. It is preferred if the polycation is polylysine.

The oligonucleotide agent may also be delivered by adenovirus wherein it is present within the adenovirus particle, for example, as described below.

In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

It will be appreciated that “naked DNA” and DNA complexed with cationic and neutral lipids may also be useful in introducing the DNA of the invention into cells of the individual to be treated. Non-viral approaches to gene therapy are described in Ledley (1995) Human Gene Therapy 6, 1129-1144.

Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell. Thus, it will be appreciated that a further aspect of the invention provides a virus or virus-like particle comprising a genetic construct of the invention. Other suitable viruses or virus-like particles include HSV, AAV, vaccinia and parvovirus.

It will be appreciated by persons skilled in the art that the agent of the invention need not be a polypeptide-based or nucleic acid-based activator of Sox11.

Thus, in an alternative embodiment the agent comprises or consists of a small molecule or a prodrug thereof.

For example, the prodrug may be selectively activated by the target cell.

The term “prodrug” as used in this application refers to a precursor or derivative form of a pharmaceutically active substance that is less cytotoxic to cancer cells compared to the parent drug and is capable of being enzymatically activated or converted into the more active parent form (see, for example, D. E. V. Wilman “Prodrugs in Cancer Chemotherapy” Biochemical Society Transactions 14, 375-382 (615th Meeting, Belfast 1986) and V. J. Stella et al “Prodrugs: A Chemical Approach to Targeted Drug Delivery” Directed Drug Delivery R. Borchardt et al (ed.) pages 247-267 (Humana Press 1985)).

Suitable methods for producing such prodrug agents are well known in the art (for example, see Denny, 2004, Cancer Invest. 22(4):604-19; Rooseboom et al., 2004, Pharmacol Rev. 2004 56(1):53-102; WO 03/106491).

In one embodiment, the agent comprises a lipoplex or a polyplex.

In a further embodiment, the agent comprises a moiety for targeting delivery of the agent to cancer cells. For example, the moiety for targeting delivery of the agent to cancer cells may be an antibody or an antigen-binding fragment thereof.

By “antibody” we include substantially intact antibody molecules, as well as chimaeric antibodies, humanised antibodies, human antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments and derivatives of the same.

By “antigen-binding fragment” we mean a functional fragment of an antibody that is capable of binding to a target epitope.

Preferably, the antigen-binding fragment is selected from the group consisting of Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]).

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

Also included within the scope of the invention are modified versions of antibodies and an antigen-binding fragments thereof, e.g. modified by the covalent attachment of polyethylene glycol or other suitable polymer.

Methods of generating antibodies and antibody fragments are well known in the art. For example, antibodies may be generated via any one of several methods which employ induction of in vivo production of antibody molecules, screening of immunoglobulin libraries (Orlandi. et al, 1989. Proc. Natl. Acad. Sci. U.S.A. 86:3833-3837; Winter et al., 1991, Nature 349:293-299) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120).

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

Antibody fragments can be obtained using methods well known in the art (see, for example, Harlow & Lane, 1988, “Antibodies: A Laboratory Manual”, Cold Spring Harbor Laboratory, New York). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

It will be appreciated by persons skilled in the art that for human therapy or diagnostics, humanised antibodies are preferably used. Humanised forms of non-human (e.g. murine) antibodies are genetically engineered chimaeric antibodies or antibody fragments having preferably minimal-portions derived from non-human antibodies. Humanised antibodies include antibodies in which complementary determining regions of a human antibody (recipient antibody) are replaced by residues from a complementary determining region of a non human species (donor antibody) such as mouse, rat of rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op, Struct. Biol. 2:593-596).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-15361; U.S. Pat. No. 4,816,567) by substituting human complementarity determining regions with corresponding rodent complementarity determining regions. Accordingly, such humanised antibodies are chimaeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, J. Mol. Biol. 227:381; Marks et a, 1991, J. Mol. Biol. 222:581; Cole et at., 1985, In: Monoclonal antibodies and Cancer Therapy, Alan R. Liss, pp. 77; Boerner et al., 1991. J. Immunol. 147:86-95).

Once suitable antibodies are obtained, they may be tested for activity, for example by ELISA.

In a particularly preferred embodiment of the first or second aspects of the invention, the agent is capable of being selectively delivered to or selectively activated by target cells.

By “selectively” we mean that the inhibitory action of the agent on the biological activity of Sox11 is preferentially exerted at or within the cancer cells (other than by local administration of the agent to the site of cancer cells).

Methods for targeting agents to particular cell types, such as cancer cells, are well known in the art (for example see Vasir & Labhasetwar, 2005, Technol Cancer Res Treat. 4(4):363-74; Brannon-Peppas & Blanchette, 2004, Adv Drug Deliv Rev. 56(11):1649-59 and Zhao & Lee, 2004, Adv Drug Deliv Rev. 56(8):1193-204).

For example, the agent may comprise a target cell specific portion.

The moiety for targeting delivery of the agent to cancer cells may recognise and bind to entities on the target cancer cell. Upon contact with the target cell, the target cell specific portion may be internalised along with the Sox11 activator portion.

The entities recognised by the targeting moiety are expressed predominantly, and preferably exclusively, on the target cancer cell. The targeting moiety may contain one or more binding sites for different entities expressed on the same target cell type, or one or more binding sites for different entities expressed on two or more different target cell types.

Preferably, the targeting moiety recognises the target cancer cell with high avidity.

By “high avidity” we mean that the target cell-specific portion recognises the target cell with a binding constant of at least K_{d}=10⁻⁶M, preferably at least K_{d}=10⁻⁹ M, suitably K_{d}=10⁻¹⁰ M, more suitably K_{d}=10⁻¹¹M, yet more suitably still K_{d}=10⁻¹²M, and more preferably K_{d}=10⁻¹⁵M or even K_{d}=10⁻¹⁸ M.

The entity which is recognised may be any suitable entity which is expressed by cancer cells. Often, the entity which is recognised will be an antigen, for example CD20 or CD22.

A third aspect of the invention provides a method of treating a cancer in a patient, the method comprising administering to the patient an agent according to the first or second aspects of the invention.

Types of cancer treatable by the methods of the invention are described above in relation to the second aspect of the invention.

Preferably, the patient is human.

Advantageously, the agent is selectively delivered to or selectively activated by the cancer cells.

By ‘treatment’ we include both therapeutic and prophylactic treatment of the patient. The term ‘prophylactic’ is used to encompass the use of a polypeptide or formulation described herein which either prevents or reduces the likelihood of cancer in a patient or subject.

The term “effective amount” is used herein to describe concentrations or amounts of compounds according to the present invention which may be used to produce a favourable change in a disease or condition treated, whether that change is a remission, a favourable physiological result, a reversal or attenuation of a disease state or condition treated, the prevention or the reduction in the likelihood of a condition or disease state occurring, depending upon the disease or condition treated.

A fourth aspect of the invention provides a pharmaceutical composition comprising an agent according to aspects one or two of the invention and a pharmaceutically acceptable excipient, diluent or carrier.

In one embodiment the pharmaceutical composition is suitable for parenteral administration. Advantageously, the pharmaceutical composition is capable of targeted delivery of the agents to the cancer cells.

The present invention also includes compositions comprising pharmaceutically acceptable acid or base addition salts of the agents of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the compounds according to the present invention.

The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

As used herein, ‘pharmaceutical formulation’ means a therapeutically effective formulation according to the invention.

As discussed above, a ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

It will be appreciated by persons skilled in the art that the agents of the invention will generally be administered in admixture with a suitable pharmaceutical excipient, diluent or carrier selected with regard to the intended route of administration and standard pharmaceutical practice (for example, see Remington: The Science and Practice of Pharmacy, 19th edition, 1995, Ed. Alfonso Gennaro, Mack Publishing Company, Pennsylvania, USA). Suitable routes of administration are discussed below, and include topical, intravenous, oral, pulmonary, nasal, aural, ocular, bladder and CNS delivery.

For example, the agents of the present invention, and pharmaceutical formulations thereof, may be delivered using an injectable sustained-release drug delivery system, such as a microsphere. These are designed specifically to reduce the frequency of injections. An example of such a system is Nutropin Depot which encapsulates recombinant human growth hormone (rhGH) in biodegradable microspheres that, once injected, release rhGH slowly over a sustained period.

Alternatively, the agents of the present invention, and pharmaceutical formulations thereof, can be administered by a surgically implanted device that releases the drug directly to the required site.

Electroporation therapy (EPT) systems can also be employed for agent administration. A device which delivers a pulsed electric field to cells increases the permeability of the cell membranes to the drug, resulting in a significant enhancement of intracellular drug delivery.

Agents can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of agent delivery is the thermo-sensitive ReGel injectable. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Agents can also be delivered orally. One such system employs a natural process for oral uptake of vitamin B12 in the body to co-deliver proteins and polypeptides. By riding the vitamin B12 uptake system, the protein or polypeptide can move through the intestinal wall. Complexes are produced between vitamin B12 analogues and the drug that retain both significant affinity for intrinsic factor (IF) in the vitamin B12 portion of the complex and significant bioactivity of the drug portion of the complex.

Preferably, the pharmaceutical formulation of the present invention is a unit dosage containing a daily dose or unit, daily sub-dose or an appropriate fraction thereof, of the active ingredient. Alternatively, the unit dosage may contain a dose (or sub-dose) for delivery at longer intervals, for example bi-weekly, weekly, bi-monthly, monthly, or longer.

The agents and pharmaceutical formulations of the present invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the agents of the invention can be administered alone but will generally be administered in admixture with a suitable pharmaceutical excipient, diluent or carrier selected with regard to the intended route of administration and standard pharmaceutical practice.

For example, the agents of the invention can be administered orally, buccally or sublingually in the form of tablets, capsules, ovules, elixirs, solutions or suspensions, which may contain flavouring or colouring agents, for immediate-, delayed- or controlled-release applications. The agents of invention may also be administered via intracavernosal injection.

Alternatively, the agents of the invention may be administered in tablet form. Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropy-lmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The agents of the invention can also be administered parenterally, for example, intravenously, intra-articularly, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 to 1000 mg per adult (i.e. from about 0.015 to 15 mg/kg), administered in single or divided doses.

Thus, for example, the tablets or capsules of the compound of the invention may contain from 1 mg to 1000 mg of active compound for administration singly or two or more at a time, as appropriate. The physician in any event will determine the actual dosage which will be most suitable for any individual patient and it will vary with the age, weight and response of the particular patient. The above dosages are merely exemplary of the average case. There can, of course, be individual instances where higher or lower dosage ranges are merited and such are within the scope of this invention.

The agents of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or ‘puff’ contains at least 1 mg of a compound of the invention for delivery to the patient. It will be appreciated that the overall daily dose with an aerosol will vary from patient to patient, and may be administered in a single dose or, more usually, in divided doses throughout the day.

Alternatively, the agents of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route.

For ophthalmic use, the agents of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agents of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or parenteral administration of the agents of the invention is the preferred route, being the most convenient.

It will be appreciated by persons skilled in the art that such an effective amount of the agent or formulation thereof may be delivered as a single bolus dose (i.e. acute administration) or, more preferably, as a series of doses over time (i.e. chronic administration).

It will be further appreciated by persons skilled in the art that the agents and pharmaceutical formulations of the present invention have utility in both the medical and veterinary fields. Thus, the methods of the invention may be used in the treatment of both human and non-human animals (such as horses, dogs and cats). Preferably, however, the patient is human.

For veterinary use, an agent of the invention is administered as a suitably acceptable formulation in accordance with normal veterinary practice and the veterinary surgeon will determine the dosing regimen and route of administration which will be most appropriate for a particular animal.

Preferred aspects of the invention are described in the following non-limiting examples, with reference to the following figures:

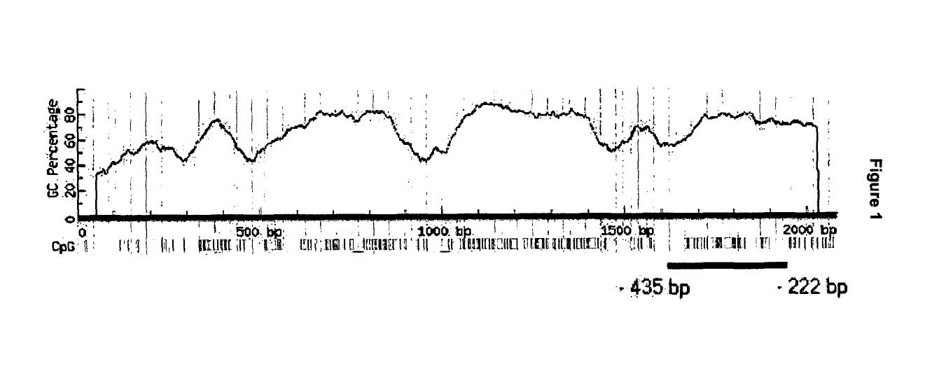

FIG. 1: CpG islands in the Sox11 promoter region

Analysis of 2000 by upstream of Sox11 transcription start revealed four CpG islands with a GC content above 50 percent (www.urogene.org/methprimer/)¹⁵. CpG dinucleotides are represented as vertical bars. Primers that amplified −435 to −222 were used in bisulfite sequencing to compare the methylation status of the Sox11 promoter region with Sox11 expression.

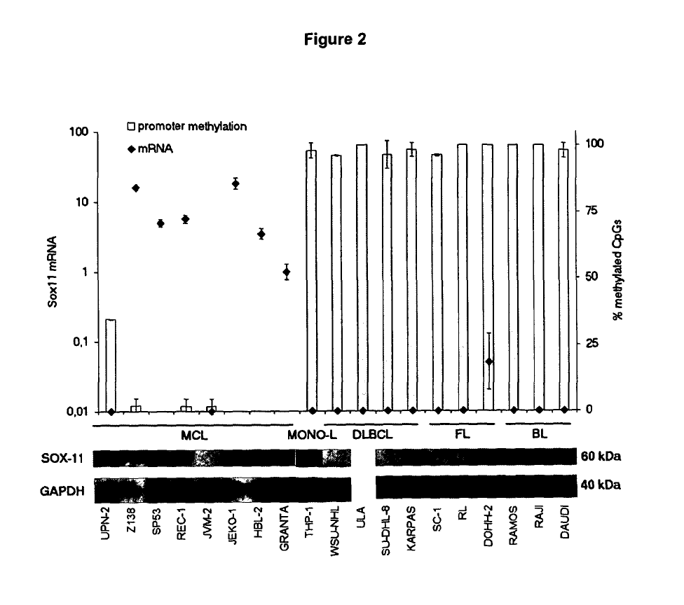

FIG. 2 NEW—Methylation status of SOX11 promoter region correlated to SOX11 expression.

Methylation status of SOX11 promoter (described as percentage of methylated CpGs of 28 possible CpG methylation sites) was analyzed by direct bisulfite sequencing (right Y-axis) and correlated to SOX11 expression on mRNA (left Y-axis) and protein level in nineteen lymphoid or monocytic cell lines (Table 2). All samples with a ΔC_{T (SOX11+RT, SOX11-RT)}<|2| was considered negative and the RQ was set to 0.01 for those samples. RQ values are related to the SOX11 expression in GRANTA-519 and the error bars show the 95% confidence interval.

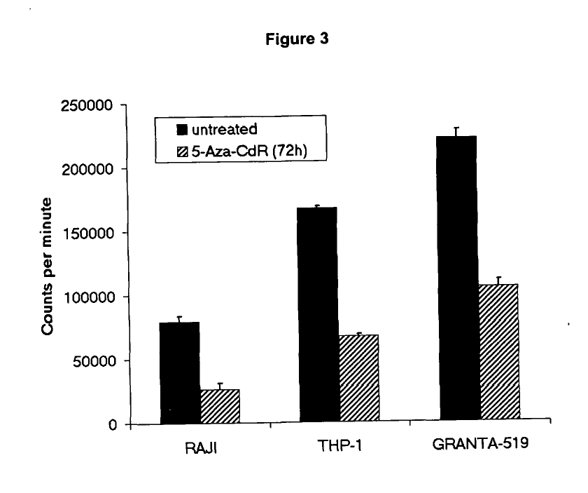

FIG. 3: Treatment with 5-Aza-CdR decreased lymphoma cell line proliferation

The demethylating agent 5-Aza-CdR caused a more than 50% decrease in proliferation rate in both methylated (RAJI and THP-1) and unmethylated (GRANTA-519) cell lines after 72 h compared to the untreated controls.

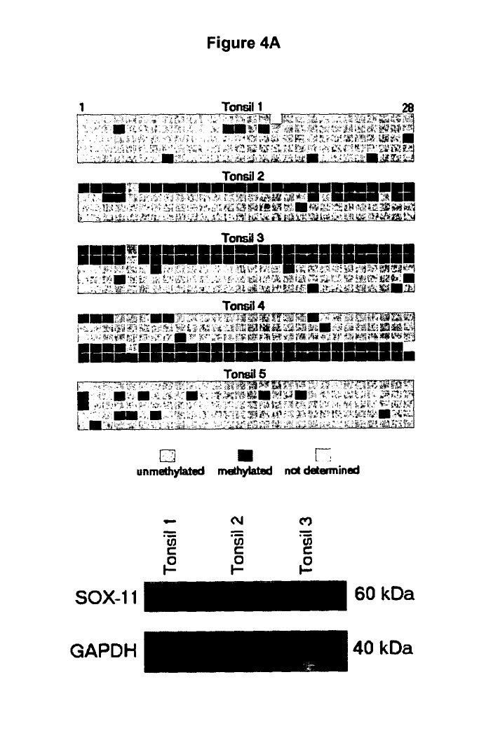

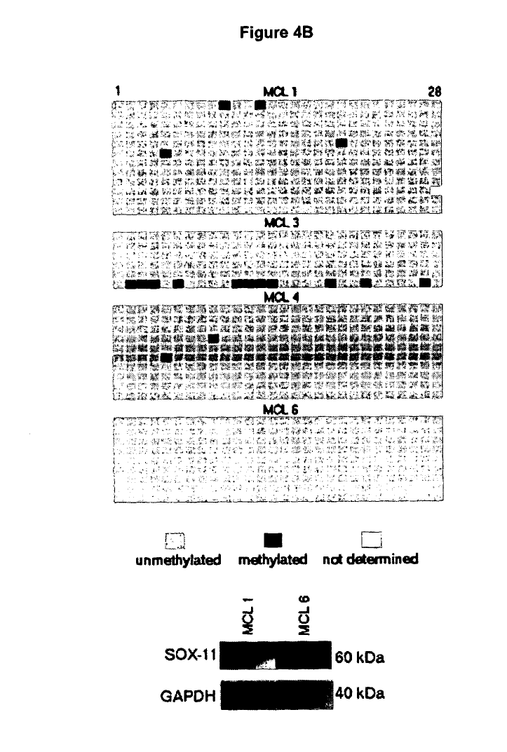

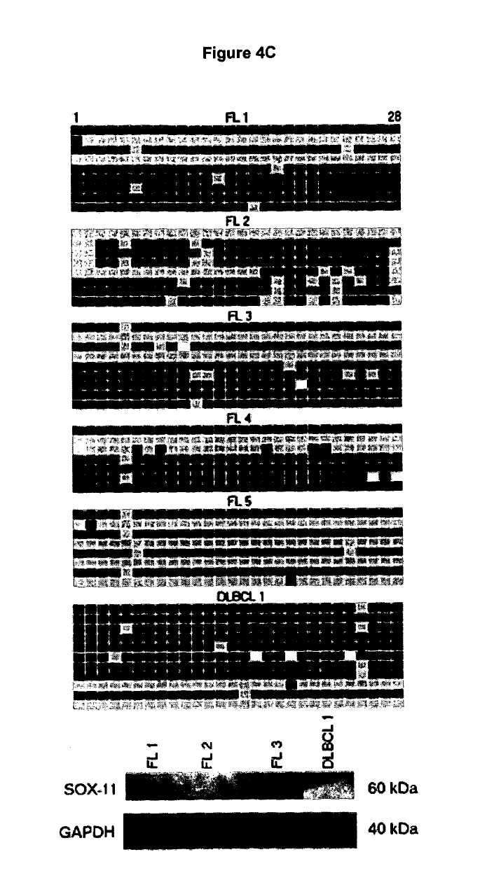

FIG. 4: Sox11 DNA methylation and protein expression in primary clinical lymphoma samples

Methylation patterns of Sox11 promoter in clinical specimens was determined by bisulfite sequencing of individual alleles and correlated to Sox11 protein expression. Every row represents a unique allele and the columns represent a potentially methylated CpG site. a) Sox11 is overall unmethylated in normal tonsil and no protein was detected. b) In MCL samples, the promoter stays unmethylated and SOX11 is detectable. c) The lack of SOX11 protein in FL and DLBCL is accompanied by >50% methylated alleles.

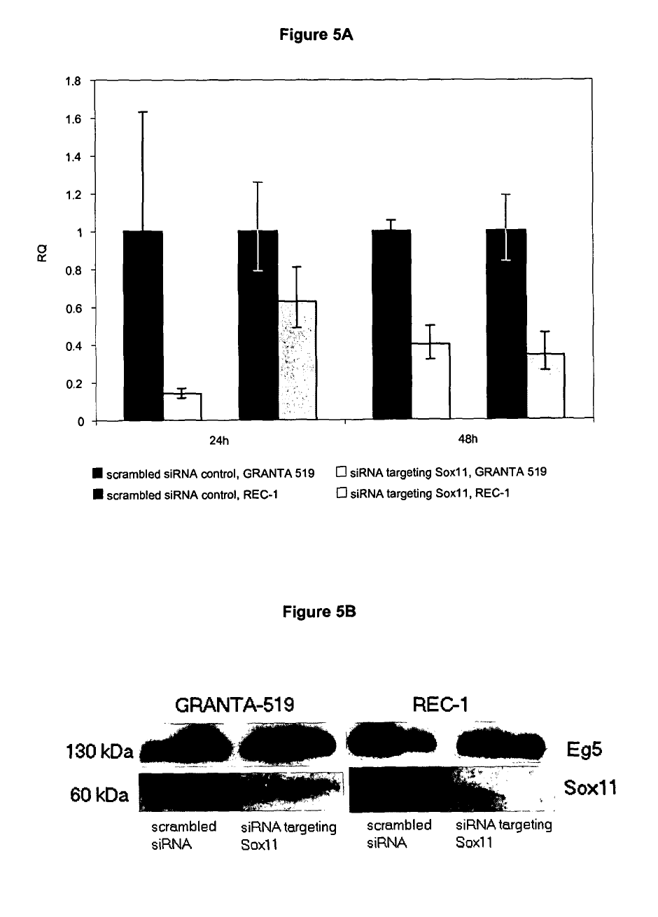

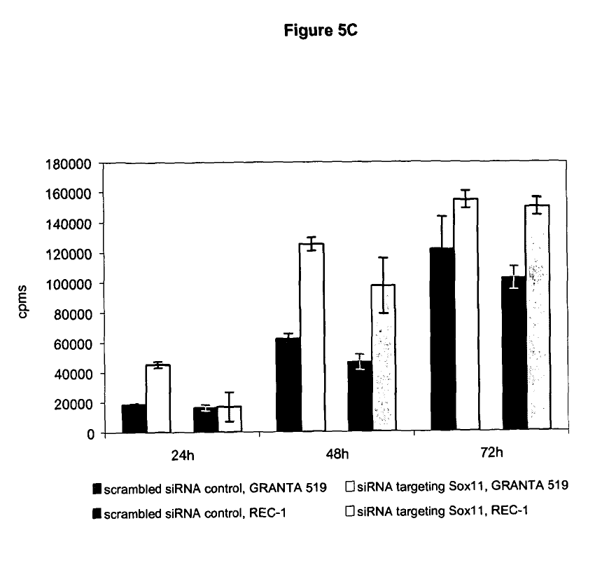

FIG. 5: siRNA knock of Sox11 increase proliferation

Effect of the siRNA induced knock-down of the Sox11 gene in GRANTA-519 and REC-1 on, a) mRNA level at 24 and 48 h; b) protein level at 72 h and 48 h, respectively, and c) proliferation at 24, 48 and 72 h. A control siRNA targeting the Eg5 gene was used as a positive control (only shown in b). All values in Figure a are relative quantity (RQ) compared to the scrambled siRNA control, which has been set to 1. The data is representative of three independent assays.

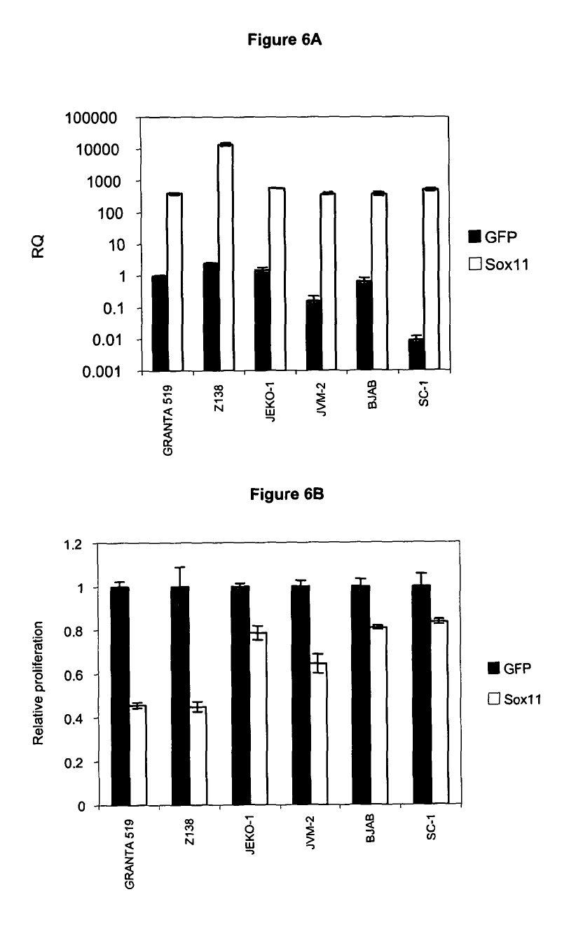

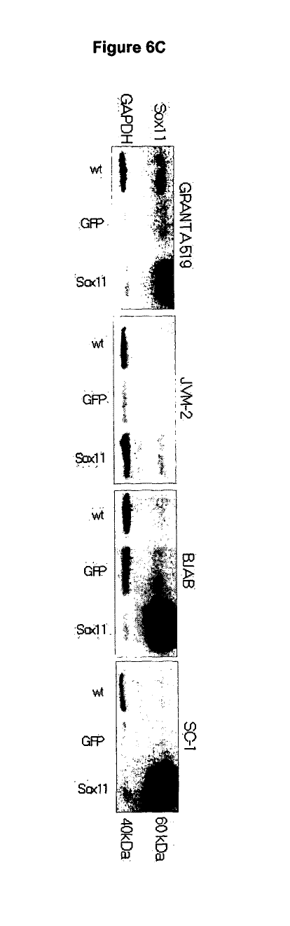

FIG. 6: Overexpression of Sox11 decrease proliferation

a) mRNA expression of Sox11 at 24 h after overexpression of the Sox11 gene in six B cell lymphoma cell lines. b) Proliferation assay at 48 h after transfection showed decreased cell growth in all cell lines, except for BJAB where the decrease could be seen already after 24 h. c) Western blot analysis at 24 h confirm Sox11 overexpression in Sox11 transfected samples (right), compared to wt (left) and control vector (middle), loading control (GAPDH) is seen below. In figure A all values are relative quantity (RQ) that have been scaled to the GFP value for GRANTA 519, which was set to 1. In figure C all cell lines are scaled to their respective GFP value, which is set to 1. The data is representative of three independent assays.

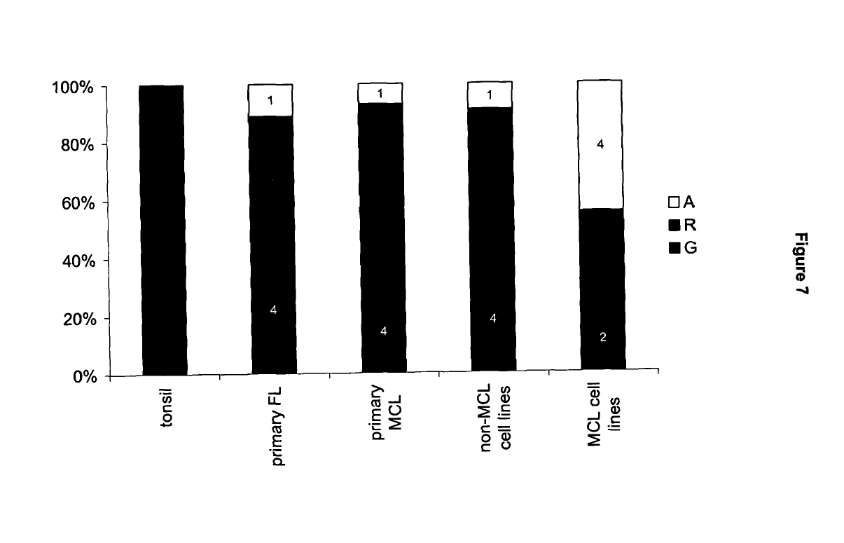

FIG. 7: SNP analysis (RS4371338) of primary and tumor cell lines

The analysis revealed that the allele usage was biased in MCL cell lines compared to non-MCL cell lines, the latter showed normal distribution as reported for Caucasians. Although not as clear, allele usage in primary MCL also seem to be biased. R-A/G

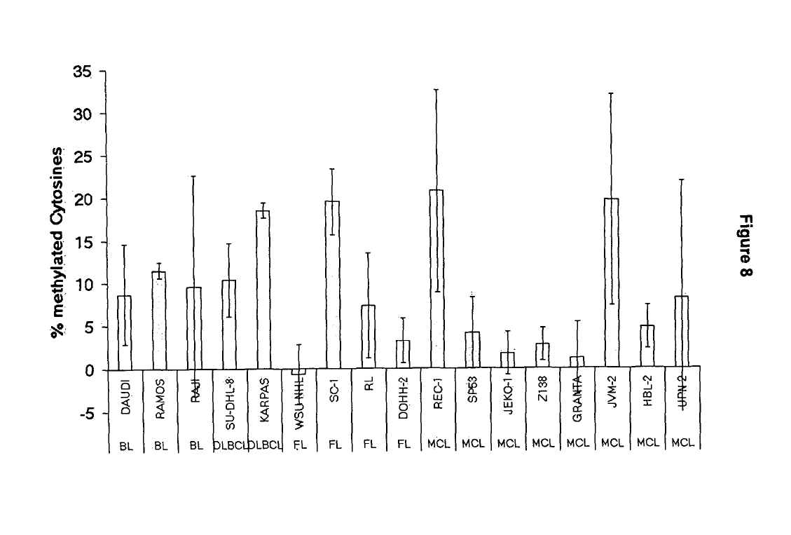

FIG. 8: Global methylation analysis of various B cell lymphoma cell lines

The analysis revealed larger variation between replicates than between samples. The experiments were repeated with kits from different vendors with similar results.

FIG. 9: Homo sapiens SRY (sex determining region Y)-box 11 (SOX11), amino acid (gi|4507161|ref|NP_{—}003099.1)

FIG. 10: Homo sapiens SRY (sex determining region Y)-box 11 (SOX11), cDNA (gi|30581115|ref|NM_{—}003108.3)

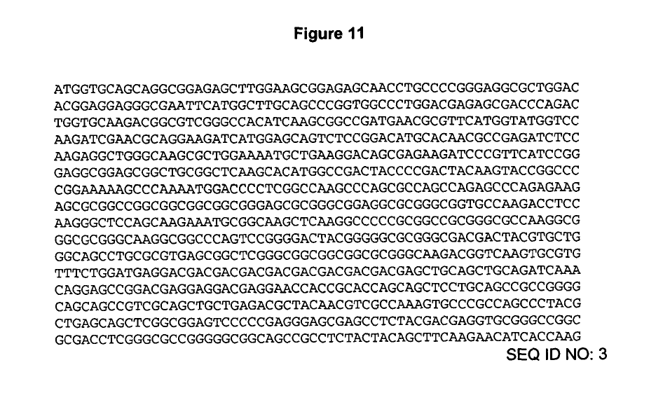

FIG. 11: CDS sequence for the OmicsLink™ Expression Clone for Sox11 (EX-M0425-M60)

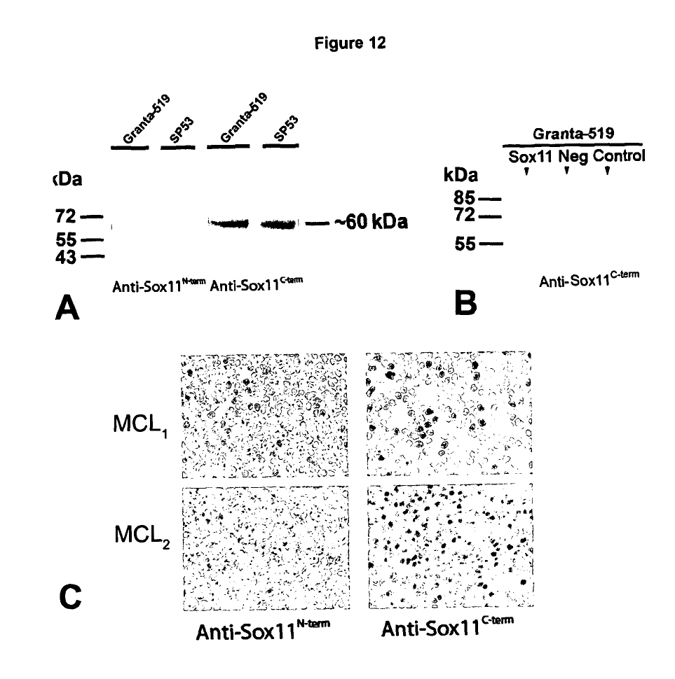

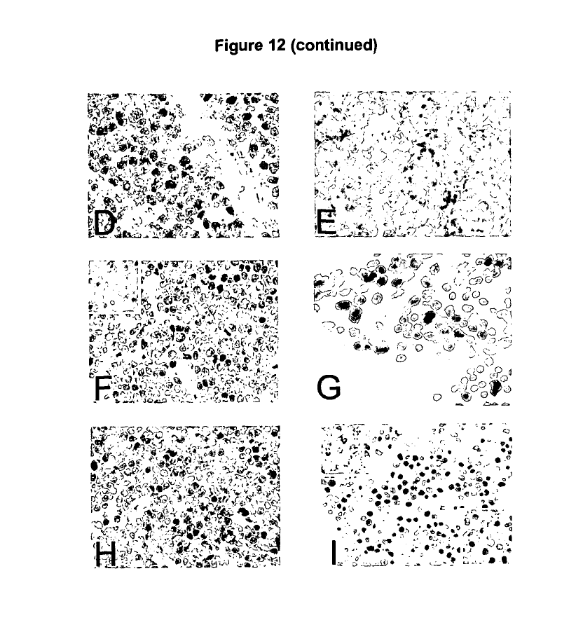

FIG. 12: (A) A Western blot of proteins extracted from two MCL cell lines shows expected ˜60 kDa bands for Sox11 using either anti-Sox11 antibody. (B) The lane labeled Sox11 denotes Granta 519 cell extract after knock-down with specific siRNA and staining with anti-Sox11^{C-term}, which yielded no band, in contrast to the Sox11 bands noted in negative and control lanes; these lanes contain extracts after nucleofection with scrambled sequence siRNA and untransfected cells, respectively. (C) A case of MCL (MCL₁) with weak nuclear signal after applying Sox11^{N-term} became stronger using Sox11^{C-term}. Another MCL (MCL₂) gave only cytoplasmic signal until immunoreacted with Sox11^{C-term}, after which nuclear signal appeared (DAB with hematoxylin counterstain, Olympus BX45, magnification ×125, colors corrected after acquisition with Adobe Photoshop).

(D) Strong nuclear Sox11 signal after staining with anti-Sox11^{C-term} is seen in a true Burkitt lymphoma. (E) Intermediate Burkitt lymphoma/diffuse large B-cell lymphoma shows no nuclear stain (signal is limited to cytoplasm). (F) Positive staining in lymphoblastic neoplasia is exemplified by a case of adult nodal T-LBL (inset, TdT stain). (G) Signal is present in a marrow with B-ALL. (H) A childhood orbital B-LBL expresses Sox11, also. (I) shows bone marrow in HCL, case 9, which expressed DBA.44 (inset, upper left), CCND1 (inset, lower right) and Sox11 with anti-Sox11^{C-term} (DAB with hematoxylin counterstain, magnification x125, except D, x230).

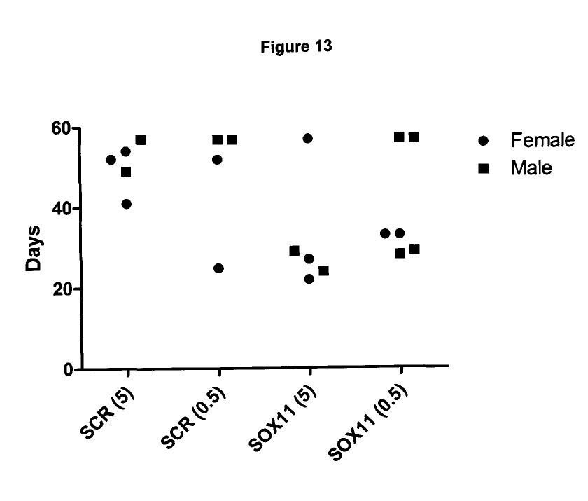

FIG. 13: Six to eight weeks old male and female NOD-SCID mice were used to assess the in vivo effect of SOX11 knock-down compared to scrambled control (scr) using either 5 or 0.5 million Z138 cells (mantle cell lymphoma cell line). Data show a shorter time (days) to death or scarification due to abnormal weight loss or other signs of tumor growth when SOX11 is knocked compared to scrambled control. The endpoint of experiment was 8 weeks after tumor cell injection at which the remaining animals (n=6) where sacrificed. In vivo data thus support a tumor suppressor function for SOX11.

### EXAMPLE A

### Introduction

The transcription factor Sox11 is a novel diagnostic marker for mantle cell lymphoma (MCL) that has recently been shown to correlate with an improved prognosis in epithelial ovarian cancer (EOC). Sox11 plays an important role in embryonic development of the central nervous system, but its extra-developmental functions remained unknown. Thus, the causes and consequences of aberrant expression of Sox11 reported for some malignancies were previously unexplained.

We show now that epigenetic regulation of Sox11 occurs in tumors as Sox11 is silenced in non-expressing malignant tissue through promoter methylation. Furthermore, for the first time we show that Sox11 directly inhibits growth in different cancer cell lines, as assessed both by siRNA-mediated knock-down and ectopic overexpression. These data demonstrate that Sox11 is not just a bystander but an active regulator of cellular growth, as ectopic over-expression of Sox11 resulted in increased proliferation of non-MCL cell lines.

### Materials and Methods

### Cultivation of Cell Lines

Twenty cancer cell lines were used to study the Sox11 gene, nine from MCL, four from follicular lymphoma (FL), three from diffused large B-cell lymphoma (DLBCL), three from Burkitt lymphoma (BL), one from acute monocytic leukemia (MONO-L) and one from B lymphoblastic lymphoma, as shown in Table 2. All cell lines were cultured in RPMI-1640 (HyClone, Sout Logan, Utah) medium supplemented with 10% (v/v) fetal bovine serum (Invitrogen Gibco, Carlsbad, Calif., USA) and 2 mM L-Glutamine (Sigma-Aldrich, St. Louis, Mo., USA), hereafter referred to as R10 medium, except ULA which was cultured in 45% optiMEM (HyClone), 45% IMDM (HyClone) supplemented with 10% (v/v) fetal bovine serum (Invitrogen).

### Gene Expression Analysis of Sox11

Gene expression values for Sox11 in the various cell lines were identified, as previously described^{1,14}. Briefly, all samples were analyzed on Affymetrix U133 plus 2.0 arrays (Santa Clara, Calif.) and MAS 5 (Affymetrix) was used to scale the arrays to an overall target value of 100. The Sox11 mRNA values shown in FIG. 2 were derived from Affymetrix internal probe id 204914_s_at.

### Sequencing of Sox11

Genomic DNA was isolated from all cell lines listed in Table 2, using QIAamp DNA MINI Kit (QIAgen, Hilden, Germany) followed by RNAse treatment (Fermentas Life Science, Ontario, Canada). Sequencing of each Sox11 exon was performed by Eurofins MWG GmbH Ebersberg, Germany) using 57 different sequence specific primers (see Table 4 for detailed list).

### SNP Analysis of RS13419910 and RS4371338

Single nucleotide polymorphisms (SNP) analyses were performed, using Sample-to-SNP kit (Applied Biosystem, Foster City, Calif., USA) and Taqman assays C_{—}32195818_{—}20 and C_{—}27292007_{—}10 corresponding to RS13419910 (dbSNP cluster id, www.ncbi.nlm.nih.goc/SNP) and RS4371338, respectively (see Supplementary data Table 5 for sequences). Briefly, 3 sections (10 μl) of paraffin-embedded tissue were deparaffinized in xylene and absolute ethanol and rehydrated using a routine protocol (see Table 6 for sample list). The Sample-to-SNP protocol was followed and samples were lyzed in designated buffer by heating to 95° C. for 3 min after which neutralization buffer was immediately added. For analysis of suspension cell cultures, 2 ml of log-phase culture were washed and pelleted. Subsequently, the cells were lyzed in designated buffer in RT for 3 min after which neutralization buffer was immediately added. 5 μl cell lysate was added to each 25 μl reaction (Taqman assay mix, master mix and DNAse free water). 40 cycles (95° C., 3s: 60° C., 30s) were performed in a 7500 FAST qPCR (Applied Biosystem).

### Collection and Purification of Primary Samples

Lymphocytes were isolated from five pediatric tonsils, four MCLs, five FLs, and one DLBCL through density centrifugation, as previously described¹⁴. Two of the tonsil samples (tonsil 4 and 5) were further purified by T cell depletion, as previously described.¹⁴ All five FL samples and two of the MCL samples (MCL1 and MCL6) were purified by positive selection, using a CD19 specific antibody (clone HD37, DAKO, Glostrup, Denmark) coupled to Dynabeads Pan Mouse IgG magnetic beads (Invitrogen Dynal), according to the protocol of the manufacturer. Flow cytometry was used to determine the purity of tonsil 4 and 5, MCL 3 and 4 and the DLBCL. All data is shown in Table 7.

### DNA Methylation Analysis

MethPrimer (www.urogene.org/methprimer/)¹⁵ was used to analyze the 2000 by region directly upstream of the SOX11 transcription start site (the SOX11 promoter region) for the presence of CpG islands. Using the MethPrimer default algorithm, three CpG islands were identified as >200 by regions with G and C contents >50% and Observed/Expected CpG-rates of >0.6. One additional CpG island was detected when the region size constraint was lowered to 100 by without altering the other criteria (FIG. 1).¹⁵ The methylation status of the 5′-promoter region was determined by sodium bisulfite sequencing.¹⁶ Briefly, total genomic DNA was extracted from five million cells per cell line or primary samples, using QlAamp DNA MINI kit (QIAgen) according to the protocol of the manufacturer. DNA concentration was determined by the Nanoprop™ (Nanoprop Technologies, Delaware, USA). To convert unmethylated cytosine to uracil, we performed bisulfite conversion of 0.5-1 μg of DNA with EpiTect Bisulfite Kit (QIAgen). The CpG island, −435 to −222 by upstream of the Sox11 transcription start site comprising 213 bp, was amplified from bisulfite converted DNA, using primers 5′-AGA GAG ATT TTA ATT TTT TGT AGA AGG A-3′ and 5′-CCC CCT TCC AM CTA CAC AC-3′. Platinum Taq DNA polymerase (Invitrogen) was used in all PCR reactions. PCR products were both directly sequenced as well as ligated into the vector pCR.21-TOPO and transformed into chemically competent E. coli TOP10. Direct sequence analysis and clonal analysis were made with primers specific for bisulfite converted DNA and vector specific primer M13(−29), respectively. All sequencing was performed by Eurofins MWG GmbH, using cycle sequencing technology on an ABI 3730XL instrument. Quality control of methylation data was performed in a standardized manner, using the BiQ Analyzer software¹⁷, (http://biq-analyzerbioinf.mpi-inf.mpg.de/index.php). Images of CpG methylation for FIGS. 4A-C were constructed using the BDPC web server¹⁸, using output files from BiQ Analyzer. All amplicons included in the study had, (i) bisulfite conversion rates above 95% for unmethylated non-CpG C:s to T:s, and (ii) sequence similarity above 90% compared to the original genomic sequence.

### Demethylation Assay

For demethylation studies, two cell lines with methylated Sox11 promotor region (RAJI and THP-1) and one unmethylated cell line (GRANTA-519) were treated with either 1 μM of 5′-Aza-2′ deoxycytidine (5-Aza-CdR, Sigma) for 72 hours alone or with 5-Aza-CdR for 72 hours followed by a 5-Aza-CdR and Trichostatin A (TsA) treatment for 24 h. Supplements of 1 μM (5-Aza-CdR) was made every 24 hour. Equivalent amount of R10 media alone were added to mock-treated cells.

### Global Methylation Determination

Global methylation analysis was performed using Methylamp Global DNA Methylation Quantification Ultra Kit (Epigentek Group Inc., New York, N.Y., USA), according to the protocol of the manufacturer. Briefly, 100 ng of DNA was immobilized in duplicates on a high affinity strip. A 5-methylcytosine specific antibody was used for detection and the enzymatic product was read at 490 nm, using an ELISA reader (Molecular Devices, Sunnyvale, Calif., USA). A universally methylated control DNA was used to create a standard curve and the percentage of methylated CpGs was subsequently calculated.

### Nucleofection

The Amaxa protocol (http://www.lonzabio.com/protocols.html) for nucleofection of suspension cell lines was followed, using program 0-017 and Cell Line Nucleofector Solution T (Amaxa Biosystems, Cologne, Germany). For the knock-down experiments, 5×10⁶ cells were mixed with 50 μmol of siRNA (Ambion, Austin, Tex., USA) in each reaction and a scrambled sequence and GFP-producing plasmid were used as controls. The sequences of the siRNAs in the pool targeting the Sox11 gene can be found in Table 3. For the overexpression experiments, 5×10⁶ cells were mixed with 2 μg of OmicsLink™ Expression Clone for Sox11 (EX-M0425-M60 sequence can be found in the FIG. 11) in each reaction and a GFP control vector was used as a control (both from GeneCopoeia, Germantown, Md., USA).

### RNA Isolation and Real Time-Quantitative PCR

In the knock-down experiments RNA isolation was carried out, using Trizol (Invitrogen,) as previously described.⁹ The cDNA synthesis was performed, as outlined in the RevertAid™ First Strand cDNA Synthesis kit-protocol (Fermentas). 1 μg of RNA was mixed with 0.2 μg random hexamer primers, and a reverse transcriptase was added to produce cDNA. Samples for real time-quantitative PCR(RT-qPCR) were prepared following the iQ™ SYBR Green Supermix protocol (Bio-Rad, Hercules, Calif., USA). The concentration of cDNA was 1.25-2.5 μg/I and the concentration of the primers was 250 nM (MWG, High-Point, N.C., USA). The primers were as follows: Sox11 (knockdown experiments): 5′-CCAGGACAGAACCACCTGAT-3′ (SEQ ID NO: 71) and 5′-CCCCACAAACCACTCAGACT-3″ (SEQ ID NO: 72), GAPDH: 5′-TGGTATCGTGGAAGGACTC-3′ (SEQ ID NO: 73) and 5′-AGTAGAGGCAGGGATGATG-3′ (SEQ ID NO: 74), Sox11 (overexpression experiments): 5′-GGTGGATAAGGATTTGGATTCG-3″ (SEQ ID NO: 75) and 5′-GCTCCGGCGTGCAGTAGT-3″ (SEQ ID NO: 76), Eg5: 5″-GTTTGGCCATACGCAAAGAT-3″ (SEQ ID NO: 77) and 5″-GAGGATTGGCTGACAAGAGC-3′ (SEQ ID NO: 78). The RT-qPCR was run in triplicate, using a 2-Step Amplification and melt-curve program (Bio-Rad) previously described⁹ with GAPDH as the endogenous control. Similarly, in the over-expression experiments, the unmodified cell lines and the demethylation assays, the Fast SYBR Green Cells-to-CT kit (Applied Biosystems) was used for lysis of the cells and cDNA synthesis, according to the protocol of the manufacturer. Briefly, 0.1−1×10⁵ cells were washed in PBS, lysed and treated with DNase. Lysates were reversed-transcribed and cDNA amplified in three technical replicates with primers specific either for Sox11 and GAPDH. q-PCR conditions were as follows: enzyme activation 20 seconds at 95° C., PCR cycle denaturation for 3 seconds at 95° C. and annealing/elongation 30 seconds at 60° C. run on a 7500 real-time qPCR system (Applied Biosystems). All samples were run in triplicates. For the unmodified cell lines, in the reverse-transcription, a control sample was run containing lysate but no reverse transcriptase (RT), to check for background amplification of genomic SOX11 and GAPDH. A ΔC_{T}>4 for GAPDH (+RT) and GAPDH (−RT) was achieved for all unmodified cell lines. Similarly, the ΔC_{T} for SOX11 (+RT) and SOX11 (−RT) was used as a qualitative control to determine if SOX11 was expressed or not in the unmodified cell lines. All samples with a ΔC_{T (SOX11+RT, SOX11-RT)}<|2| were considered negative and the RQ was set to 0.01 for those samples. Finally, RQ is calculated as 2^{-(ΔΔCT(SOX11-GAPDH))} comparing each cell line to GRANTA-519. The error bars related to qPCR data were calculated using standard error (SE) with a 95% confidence level.

### Protein Purification and Quantification

72 hours post-nucleofection, 0.5−2×10⁶ cells were harvested, washed and placed in 200 μl lysis-buffer (1% NP40/Protease Inhibitor cocktail (Roche, Basel, Switzerland) in PBS) and incubated on ice for 30 min. Centrifugation (16,000×g at 4° C. for 30 min) was used to remove cell debris. Protein concentrations were determined using the BCA Kit for Protein Determination (Sigma) with BSA as a standard (0.08-0.4 mg/ml). The samples were mixed with BCA working reagent, incubated at 37° C. for 30 min, and absorbance measured at 562 nm. Protein lysates for western blot analysis were prepared from 0.5−1×10⁶ cells as above.

### Western Blot Analysis of Sox11-Knockdown and Differential Expression

Protein lysates, 3 or 7 μg for knock-down experiments, 3.5 μg for overexpression experiments and 32 μg for wild-type expression in nineteen lymphoma cell lines and fifteen excised specimens were run on NuPAGE 10% Bis-Tris gels (Invitrogen) under reducing conditions for ˜45 min at 130 V. Separated proteins were blotted onto PVDF membranes, Amersham Hybond-P (GE Healthcare, Uppsala, Sweden) for 30 min (15 V) and blocked over night in 5% milk PBS. Sox11 protein expression was verified using Sox-11^{C-term} (FIG. 2-5) or Sox-11^{N-term} (FIG. 6), as previously described.^{1,19} Primary antibodies Eg5 (Becton Dickinson, N.J., USA) or GAPDH (Abcam) were used as loading control. HRP-labeled swine anti-rabbit antibody or rabbit anti-mouse antibody (DAKO) was used as secondary antibody and detection was made with SuperSignal West Femto Max Sensitivity Substrate (Pierce), according to the protocol of the manufacturer. Blots were developed, using the SuperSignal West Femto Maximum Sensitivity Substrate (Nordic Biolabs, Täby, Sweden) on ECL Hyperfilm (GE Healthcare) in Kodak X-OMAT 1000 processor (Kodak Nordic AB, Upplands Vasby, Sweden).

### Assessment of Proliferation in B Cell Lymphoma Cell Lines Upon Alteration of Sox11 Content and 5-Aza-CdR

All proliferation assays were quantified using Methyl-3H-Thymidine (MTT) incorporation, as previously described.²⁰ 50 000 cells were plated in triplicates for each sample. For all proliferation results, the ±1 standard deviation (SD) is shown.

### Results

### Difference in Allele Usage Between MCL and Non-MCL Cell Lines

Aberrant expression of genes can have varied causes, including mutations in both the coding sequences and 3′-UTR of mRNAs.²¹ We set out to investigate the potential difference in Sox11 gene sequence, both coding and non-coding, comparing Sox11 positive and negative tissue and cell lines.

Two coupled and recurrent polymorphisms were identified in Sox11 through sequencing of 20 different B cell lymphoma cell lines (see Table 2). These two SNPs were located at position 5732 by and 7388 bp, in the untranslated 3′ region, and corresponded to the defined SNPs RS13419910 and RS4371338, respectively (see Table 5). Taqman assays (C_{—}32195818_{—}20 and C_{—}27292007_{—}10) targeting these two SNPs gave results identical with direct sequencing for four analyzed cell lines. Subsequently these Taqman assays were used to screen primary tonsil, FL and MCL samples. In general, C_{—}27292007_{—}10 gave a more clear prediction than C_{—}32195818_{—}20. As previously seen using sequencing, the two SNPs were coupled and gave corresponding results for all samples analyzed (see Table 2). Analysis of these two polymorphisms indicated that there is a difference in allele usage comparing MCL and non-MCL cell lines, although the statistical significance is weak (Pearson chi-square test, p=0.1921). The distribution of the non-MCL cell lines corresponded to the normal distribution reported for Caucasians (45%, 45%, 10%, (www.ncbi.nlm.nih.goc/SNP, dbSNP cluster id). Subsequent analysis of a small material of primary samples showed no difference comparing FL and MCL tumors (FIG. 7).

### Methylation Status of Sox11 Promoter Region Correlates to Protein Expression in Lymphoma Cell Lines

To further assess the regulation of Sox11 expression in malignant lymphoid tissue and cell lines, epigenetic regulation, as assessed by promoter methylation analyses, of Sox11 expression was investigated.

Analysis of 2000 by region upstream of the transcription start site of Sox11 identified four CpG islands (FIG. 1). DNA hypermethylation of such islands is a common event in tumor progression and leads to silencing of the corresponding gene.²⁸ The methylation status of the Sox11 promoter in nineteen cell lines, originating from different B cell malignancies, including eight MCL, three DLBCL, four FL, three BL and one acute monocytic leukemia (MONO-L), (Table 2) was determined using bisulfite sequencing.

Bisulfite sequencing was performed on the CpG island adjacent to the Sox11 transcription start site, covering 28 unique CpG sites (FIG. 1). One set of primers, which amplified both methylated and unmethylated sodium bisulfite converted DNA, were used. To asses the quality of bisulfite conversion and sequencing, two different quality measurements were employed by the BiQ Analyzer software. All amplicons included in the study had, (i) bisulfite conversion rates above 95% for unmethylated non-CpG C:s to T:s, and (ii) sequence similarity above 90% compared to the original genomic sequence. The amplicons were directly sequenced to give an average of the degree of methylation in the cell populations and Sox11 expression on the mRNA and protein level was verified through previous gene chip data, as well as on western blot analysis of corresponding cell lines.

A striking difference in SOX11 promoter methylation was detected between MCL and non-MCL lymphoma cell lines (FIG. 2). The results were confirmed on individual alleles with TOPO-TA cloning for seven of the cell lines (Table 1). Analysis of non-MCL cell lines revealed high levels of SOX11 promoter methylation in all cases (11/11), corresponding to a lack of both SOX11 mRNA and protein expression (FIG. 2). In contrast, SOX11 promoter methylation was absent in the majority (7/8) of MCL-derived cell lines, with SOX11 mRNA and protein expression being evident in 6 of the cell lines (GRANTA-519, HBL-2, JEKO-1, REC-1, SP53 and Z138) (FIG. 2). UPN-2 was partially methylated, and lacks SOX11 expression. JVM-2 was the only MCL cell line lacking SOX11 mRNA and protein, although the promoter was not methylated in any of the 28 CpG's investigated but did not express Sox11 protein or mRNA. To rule out the possibility that the Sox11 promoter in non-MCL cell lines could have been methylated through a non-specific increase in overall genomic methylation, ELISA-based assays to quantify global DNA methylation were performed. These global methylation analyses were repeatedly performed using reagents from different vendors, all generating data with high standard deviations. However, the variation between different cell lines of a specific tumor entity was larger than the difference between tumor entities and, thus, we concluded that the methylation of Sox11 could not be related to the overall methylation status of the cell line (see FIG. 8). Consequently, the Sox11 promoter region is specifically methylated in non-MCL lymphoma cell lines.

The promoter methylation analyses thus suggest that Sox11 expression can be epigenetically silenced in vitro. We were therefore interested in investigating if Sox11 expression could be reactivated through global demethylation of Sox11-negative cell lines. Therefore, two Sox11-negative B cell lymphoma cell lines (THP-1 and RAJI) were treated with the demethylating agent 5-Aza-CdR alone, or in combination with TsA which prevents histone deacetylation enzymes from removing acetyl groups in transcriptionally active histones.²² 5-Aza-CdR had a strong influence on the growth of the cell lines; the proliferation rates of treated cell lines were decreased by over 50% compared with untreated controls (FIG. 3). However, methylation analysis of bisulfite-converted DNA extracted from treated cells revealed that the Sox11 promoter methylation was unaffected by these agents (data not shown), potentially due to poor proliferation, and consequently no Sox11 expression was induced, as determined with qPCR using the corresponding untreated cell lines and the Sox11-positive GRANTA-519 cell line as controls. These experiments were repeated twice with the same results.

Levels of Sox11 Protein in Malignant and Non-Malignant Clinical Specimens Show Correlation with Promoter Methylation Status

Untreated clinical specimens were collected to assess the methylation status in non-malignant B cells (tonsil, n=5), primary MCL (n=4), FL (n=5) and a single case of DLBCL (see Table 7). Most samples were purified, using either CD3-depletion or positive selection on CD19-coated beads (see Table 7). Flow cytometry analysis of tonsil 4 and 5 showed a highly purified B cell population, with >95% CD19 positive cells, while MCL 3 and 4 showed a purity of between 80 and 96%, respectively, with the CD3+population only constituting 2-3% in both cases (Table 7). Thus, the analyzed samples constitute predominately B cells. Nevertheless, the frequency of tumor cells within the pure B cell population will vary among entities.

Overall, DNA isolated from normal B cells was unmethylated in the Sox11 promoter region (FIG. 4A). Samples 1 and 5 displayed sporadic methylation, while a few completely methylated alleles were detected in tonsil 2, 3 and 4. Nevertheless, independent of the methylation status of the promoter, no Sox11 protein could be detected in normal B cell samples (FIG. 4A). In agreement with the data on the in vitro models of B cell lymphoma, the Sox11 promoter region is also unmethylated in primary MCL (FIG. 4B) consistent with the protein analysis of the tested material (FIG. 4B lower panel). Furthermore, extensive DNA methylation was seen in one DLBCL and most FL, apart from FL1 where less than 50% of the alleles are methylated, possibly due to contamination with non-malignant B cells (FIG. 4C). As expected, none of the tested non-MCL subtypes were positive for Sox11 protein (FIG. 4C). Consequently, the lack of methylation in normal tonsil and MCL compared to the methylated state of FL and DLBCL samples, points towards specific Sox11 silencing due to hypermethylation in all Sox11-negative B cell lymphomas analyzed.

### Sox11 Knockdown in MCL Cell Lines is Accompanied by Increased Cell Proliferation

Previous studies, where Sox11 has been correlated to improved overall or recurrence free survival^{1,7}, indicate that Sox11 might regulate tumor cell growth. Thus, to further investigate this, we assessed the functional effect of Sox11 on cellular proliferation using well characterized in vitro models of MCL (GRANTA-519 and REC-1), as well as a Sox11-negative FL cell line, RL, as a control. Transient silencing of Sox11 expression, using nucleofection and specific siRNA (see Table 3), mediates a significant decrease at both mRNA (FIG. 5A) and protein levels (FIG. 5B), resulting in a significant increase in proliferation of >50%, already after 24 hrs (FIG. 5C). The follicular lymphoma cell line do not express any Sox11 and no change in proliferation was consequently detected (data not shown). The effect on mRNA expression reached a maximum decrease already at 24 hrs for GRANTA-519 and at 48 h for REC-1 (FIG. 5A), while the subsequent decrease in protein level was most pronounced at 72 h for GRANTA-519 and at 48 h for REC-1 (FIG. 5B). The functional effect on cell proliferation showed an increase by >50% at 48 h for both MCL cell lines (FIG. 5C), confirming a growth modulating role for Sox11.

### Sox11 Overexpression in Sox11-Negative Cell Lines Inhibits Proliferation

As the increase in proliferation, seen following Sox11 knock-down, could be due to indirect effects, e.g. Sox11 being the limiting factor in a signaling pathway, the direct effect of Sox11 on proliferation was investigated using overexpression of Sox11 in both positive and negative cell lines (see Table 2). A suitable plasmid vector containing both the coding sequence of Sox11 under the control of a CMV promoter (see FIG. 11) was introduced through nucleofection. A vector containing GFP was used as control. Varying degrees of mRNA overexpression were evident at 24 h (FIG. 6A) for all cell lines analyzed, both cell lines originally negative (SC-1, JVM-2) as well as positive for Sox11 (BJAB, JEKO-1, GRANTA-519 and Z138). Of note, some of the originally Sox11-negative cell lines showed overexpression of Sox11 mRNA which was several thousand times that of wild-type levels. No direct correlation between mRNA and protein levels could be seen, in fact BJAB showed the strongest increase in protein level (FIG. 6C), although the increase in mRNA was among the lower (still 100 times overexpression). Conversely, JVM-2 only displayed a weak increase in protein level although the mRNA level increased by 3000 times. All cell lines showed variable overexpression of Sox11 protein (FIG. 6C), although the low amount of protein produce poor WB data quality for Z138 and JEKO-1 (data not shown). However, upon measurement of proliferation at 24 and 48 h it was clear that all cell lines grew significantly slower due to Sox11 overexpression, with a most pronounced effect at 48 h for all cell lines but BJAB, in which decreased proliferation was seen at 24 h (FIG. 6B). The strongest effect on proliferation was seen for GRANTA-519, Z138 and JVM-2 (FIG. 6B). Thus, Sox11 directly regulates growth in all cell lines analyzed independent of their original Sox11 status. As the overexpression of Sox11 was transient, no overexpression or functional effect was seen at day 6. In fact, selection with antibiotics for 6 days caused cells with forced Sox11 expression to die in contrast to cells transfected with GFP control vector (data not shown), indicating that Sox11 not only leads to slower proliferation but also permits induction of cell death.

### Discussion

Through sequence analysis of in vitro models of B cell lymphomas we identified two SNPs in the 3′UTR of Sox11 that were overrepresented in MCL cell lines, compared to other B cell lymphoma cell lines, although the difference was statistically weak. It has been shown that polymorphism also in the 3′-UTR may affect transcription level²¹, and this might be one of several explanations for the aberrant expression of Sox11 in MCL.

More commonly, a cell may regulate expression of a certain gene by an epigenetic mechanism such as DNA methylation of CpG islands in the promoter region where methyl groups are added to CpG-cytosines by methyltransferases (DNMT1, DNMT3a and DNMT3b). These sites are not evenly distributed in the genome, but are found in CpG-dense areas called CpG islands, located in the 5′ promoter region of many genes.^{23,24} In most cells, these islands are generally hypomethylated²⁵ but can become methylated in a tissue specific manner²⁶ to specifically repress the target genes.²⁷ Methylation mediated silencing of various genes, most often tumor suppressor genes, is a well studied phenomenon in many cancers²⁸ and an increasing number of hypermethylated genes have been reported in lymphomas²⁹⁻³⁶. These genes are involved in various cellular functions such as cell cycle control²⁹, cytokine signaling³³, DNA repair and apoptosis.³⁴

Analysis of the Sox11 promoter identified the presence of CpG islands, and bisulfite conversion followed by direct sequencing or sequencing of individual clones demonstrated a strong correlation between promoter methylation status and Sox11 mRNA and protein levels in both B cell lymphoma cell lines and primary tumors. Thus, as also previously reported, data from cell lines represent the methylation status of primary tissue rather well.³⁷ However, as our experiment illustrates and reported by others, the magnitude of methylation is increased in cell lines, since they are either fully methylated or unmethylated.³⁸ Altogether, it is clear that the absence of SOX11 expression is tightly coupled to a methylated promoter in primary tumor samples.

In addition to investigating the cause of the aberrant Sox11 expression, we also explored the relation between Sox11 expression and cellular growth, as a correlation with survival had been reported^{1,2}. The function of Sox11 outside the CNS remains unknown. Sox11 function in the CNS has previously been assessed, using siRNA in a mouse neuroblastoma cell line and in cultured mouse dorsal root ganglia neurons, where Sox11 was shown to modulate the levels of several other unrelated mRNAs involved in cell survival and death by increasing expression of the pro-apoptotic gene BNIP3 and decreasing expression of the anti-apoptotic gene TANK for example.⁴¹ In contrast, SOX11 was recently shown to prevent gliomagenesis in vivo by induced neuronal differentiation and abolished expression of oncogenic plagl1.⁴⁶ Recent clinical studies have shown both a positive and negative correlation of SOX11 to survival and further studies have consequently been lacking to fully explore the clinical implications of this marker.^{47, 2, 4} In the present study, transient knock-down experiments confirm a tumor suppressor function for Sox11, as decreased levels induce increased proliferation in several in vitro models of MCL. To further clarify if Sox11 is the limiting factor in a signaling cascade or if Sox11 possibly exhibits master regulatory properties, we overexpressed Sox11 in various B cell lymphoma cells lines with variable degree of wild-type Sox11 expression. Overexpression was achieved in all cell lines, independent of the original Sox11 status, and was reflected by a variable increase in Sox11 protein. Of note, all cell lines were functionally affected and their growth rates were significantly reduced. The direct effect on proliferation upon increasing the Sox11 level confirms that Sox11 is a master regulator and that the functional effect of Sox11 is not specific for MCL but can be induced upon expression in other B cell lymphomas.

Thus, SOX11 appears to have an opposite effect in B cell lymphomas and gliomas⁴⁶ compared to the normal murine CNS,⁴¹ which could be due to binding of different transcription factor partners. Previous work has suggested that gene expression in a specific cell is influenced by the specific combination of POU (pic, oct and unc transcription factor families) and SOX family members¹⁰ and it is not unlikely that SOX11 can act both as a tumor suppressor and oncogene depending on the cellular context and protein partners, as have been reported for SOX4^{42, 43} and several other transcription factors.^{44, 45}

In summary, we have for the first time shown that the expression of Sox11 is regulated through specific promoter methylation. Furthermore, we demonstrate that Sox11 has a tumor suppressor-like function and a master regulator of tumor cell growth. We have for the first time shown that the expression of the transcription factor SOX11 is inversely correlated to specific promoter methylation in hematopoietic malignancies and that SOX11 has a tumor suppressor-like function. Thus, based on both experimental and previous clinical observations this indicates that SOX11 acts as a master regulator of lymphoid tumor cell growth.

TABLE 2
Cell lines included in the study, the SNP status and
DNA-methylation analyses performed

| | | | | | DNA- |
|---|---|---|---|---|---|
| | | | | | methyl- |
| | Lym- | | SNP | SNP | ation |
| Cell line* | phoma** | Supplier | RS4371388 | RS13419910 | analysis |
| GRANTA- | MCL | DSMZ | A/G | A/G | D, T |
| 519 | | | | | |
| SP53 | MCL | ***** | A | G | D, T |
| Z138 | MCL | **** | A/G | A/G | D, T |
| HBL-2 | MCL | | A | G | D |
| JEKO-1 | MCL | DSMZ | G | A | D |
| JVM-2 | MCL | DSMZ | A/G | A/G | D |
| REC-1 | MCL | DSMZ | A | G | D |
| UPN-2 | MCL | | A | G | D, T |
| NCEB-1 | MCL | ATCC | G | A | |
| BJAB | Lympho- | | G | A | |
| | blastoid | | | | |
| WSU-NHL | FL | DSMZ | G | A | D, T |
| SC-1 | FL | DSMZ | A/G | A/G | D |
| RL | FL | DSMZ | G | A | D |
| DOHH-2 | FL | DSMZ | A | G | D |
| SU-DHL-8 | DLBCL | *** | G | A | D, T |
| ULA | DLBCL | *** | — | — | D |
| KARPAS | DLBCL | *** | A/G | A/G | D |
| RAMOS | BL | DSMZ | A/G | A/G | D |
| RAJI | BL | DSMZ | A/G | A/G | D |
| DAUDI | BL | DSMZ | A/G | A/G | D |
| THP-1 | MONO-L | DSMZ | A/G | A/G | D |

*The Sox11 gene was sequenced in all cell lines
**see text for abbreviations
*** Kindly provided by Dr Kristina Drott, Lund University
**** Kindly provided by Dr Dyer at Leicester University
***** Kindly provided by Dr Mats Ehinger, Lund University
D direct sequencing
T TOPO-TA cloning of individual alleles
DSMZ, Deutsche Sammlung von Mikroorganismen and Zellkulturen GmbH
ATCC, American Tissue and Culture Collection

TABLE 3
Sequences of? the siRNAs targeting Sox11^{a}

| Sequence | Sense 5′→3′ | Antisense 5′→3′ |
|---|---|---|
| Sox11.1 | CAAGUAUGUUGGUACGUUAuu | UAACGUACCAACAUACUUGuu |
| (pool) | SEQ ID NO: 4 | SEQ ID NO: 8 |
| | GAUAAGAUGUCGUGACGCAuu | UGCGUCACGACAUCUUAUCuu |
| | SEQ ID NO: 5 | SEQ ID NO: 9 |
| | CCUCUAGGCUCCUCGAAGAuu | UCUUCGAGGAGCCUAGAGGuu |
| | SEQ ID NO: 6 | SEQ ID NO: 10 |
| | GUUUGAAGCUUGUCGGUCUuu | AGACCGACAAGCUUCAAACuu |
| | SEQ ID NO: 7 | SEQ ID NO: 11 |

^{a}nucleotides written in small letters are overhangs

TABLE 4
Primers used for Sox11 sequencing

| Primer | Sequence | |
|---|---|---|
| sox11-1f | AAA GCG GGG TGC CGA GGA CT (20 bp) | SEQ ID NO: 12 |
| sox11-1r | CTT GAG CTT GCC GCA TTT CTT G (22 bp) | SEQ ID NO: 13 |
| sox11-2f | AGC CAG AGC CCA GAG AAG AGC (21 bp) | SEQ ID NO: 14 |
| sox11-2r | CTG CTG GAC GAG GAG GTG GA (20 bp) | SEQ ID NO: 15 |
| sox11-3f | GCC GCC TCT ACT ACA GCT TCA AGA (24 bp) | SEQ ID NO: 16 |
| sox11-3r | CAA TTT CTT TGC GTC ACG ACA TCT (24 bp) | SEQ ID NO: 17 |
| sox11-4f | CCT TGG GAG GAA GTT GTA GTG GTG (24 bp) | SEQ ID NO: 18 |
| sox11-4r | CAC ATT TGT AAA ACC ATA AAC AAT TTG A (28 | SEQ ID NO: 19 |
| | bp) | |
| sox11-5f | TTG GAG GGA GAA AAC TGA TGT CTT (24 bp) | SEQ ID NO: 20 |
| sox11-5r | CCA TCC ACA TCA CAG CGT ATG AGA (24 bp) | SEQ ID NO: 21 |
| sox11-6f | TGA AAA TGG TGA TAT AGA CCT CAG AGC (27 | SEQ ID NO: 22 |
| | bp) | |
| sox11-6r | AAG AAC ACC CTT CCC CTG TCT TTC (24 bp) | SEQ ID NO: 23 |
| sox11-7f | TTT AGG GGG TTA GGC TGA AAA GTG (24 bp) | SEQ ID NO: 24 |
| sox11-7r | AAG GAA ACA GAC ACC GAC CAC TTC (24 bp) | SEQ ID NO: 25 |
| sox11-8f | CGT GTG CTC AGA GGT GGT TGT T (22 bp) | SEQ ID NO: 26 |
| sox11-8r | TCC CGG AGA ACA ATC AAG ATG C (22 bp) | SEQ ID NO: 27 |
| sox11-9f | CTG CGG GGT GAG AGG AAG AAA GC (23 bp) | SEQ ID NO: 28 |
| sox11-9r | GGG TGG TGG TAA GAT CGA GTA AGG (24 bp) | SEQ ID NO: 29 |
| sox11-10f | GGT TTG GCC TTC CAT TTT TAC TGA (24 bp) | SEQ ID NO: 30 |
| sox11-10r | CCT CAC CAC AGA AAA TGT CCA AGA (24 bp) | SEQ ID NO: 31 |
| sox11-11f | TTG GCA ACG TAA ACC CAT TGA TAG (24 bp) | SEQ ID NO: 32 |
| sox11-11r | GCT TAC CAA AAT GCC ATC AGA GTC (24 bp) | SEQ ID NO: 33 |
| sox11-12f | ACA CAT GGT ATT CTT GCC ACT GGA (24 bp) | SEQ ID NO: 34 |
| sox11-12r | TCT CAA ATT CCT TGG GCA AAA GTC (24 bp) | SEQ ID NO: 35 |
| sox11-13f | TTC TCT TCT GGG ACT TGA AAT CAT (24 bp) | SEQ ID NO: 36 |
| sox11-13r | CAT GGA GAC GGT TAC TTT GGG AAC (24 bp) | SEQ ID NO: 37 |
| sox11-14f | CCC TTT GTA TAG CCT AAG CCT GTG A (25 bp) | SEQ ID NO: 38 |
| sox11-14r | TGC ACT GGC AGA GGT GCT AGA T (22 bp) | SEQ ID NO: 39 |
| sox11-15f | CGG CTT ACA AAG GGA GAC ACA AGC (24 bp) | SEQ ID NO: 40 |
| sox11-15r | ATG TGA TTC AAG GGA GGA GGC ATA (24 bp) | SEQ ID NO: 41 |
| sox11-16f | CAC GTT ACA TTT CCC CTT CCA AAA (24 bp) | SEQ ID NO: 42 |
| sox11-16r | GCT ATC AAA CAC TTC ATC CTC CAG (24 bp) | SEQ ID NO: 43 |
| sox11-17f | TGT GTA GAA GTC TGA GTG GTT TGT GG (26 bp) | SEQ ID NO: 44 |
| sox11-17r | ATC TTC AAG CCT GTC CCT GAC ATC (24 bp) | SEQ ID NO: 45 |
| sox11-f1b | AAC TTG CCC AGG AAG GTG (18 bp) | SEQ ID NO: 46 |
| sox11_f2b | GTG CCA AGA CCT CCA AGG (18 bp) | SEQ ID NO: 47 |
| sox11_r2b | TGC TGC TTG GTG ATG TTC (18 bp) | SEQ ID NO: 48 |
| f10n1a | AGC GTC CGC ACA GTA AC (17 bp) | SEQ ID NO: 49 |
| f10n1b | CCC TTC TTT TCC CAA ATG (18 bp) | SEQ ID NO: 50 |
| f10n2a | GAT GCG AAG CCA GCA AG (17 bp) | SEQ ID NO: 51 |
| f10n2b | ACC TCA CCA CAG AAA ATG TC (20 bp) | SEQ ID NO: 52 |
| f12n1a | TCT GAT GGC ATT TTG GTA AG (20 bp) | SEQ ID NO: 53 |
| f12n2a | AAA AAA AAA AAT GCT AAT AAA AG (23 bp) | SEQ ID NO: 54 |
| f12n3a | TTT TTT TTA AAT AAA AGG GAT G (22 bp) | SEQ ID NO: 55 |
| 16fb | CCC TTC CAA AAA AAA AAA AAA G (22 bp) | SEQ ID NO: 56 |
| 15rb | GTT GTC CAA AAA AAA AAA AAA C (22 bp) | SEQ ID NO: 57 |
| 6if | GCA AAA AAG AAA AAA AAA AG (20 bp) | SEQ ID NO: 58 |
| 6ir | CCT TTT TTT TTT CTT TTT TGC (21 bp) | SEQ ID NO: 59 |
| sox11_r10b | CTT CCC ATT CTG AAG CCA AA (20 bp) | SEQ ID NO: 60 |
| f4b | TTT TTT TTT TGG AGG G (16 bp) | SEQ ID NO: 61 |
| r4b | TTT TTT TTT TGT AAG CG (17 bp) | SEQ ID NO: 62 |
| f5i1 | GTT GGT TTA AAA AAA AAA AGC (21 bp) | SEQ ID NO: 63 |
| f6i2 | GCC TGT TTT TTT TTT TTT TTT TTT GTG (27 | SEQ ID NO: 64 |
| | bp) | |
| f11i1 | GTC AAG ATT TTT TTT TTT TAA AGC (24 bp) | SEQ ID NO: 65 |
| f15i1 | GTC CTT TTT TTT TTT TTT GG (20 bp) | SEQ ID NO: 66 |
| f14i1 | TTT TTT TTT TTT TTC CTT G (19 bp) | SEQ ID NO: 67 |
| r14i1 | AAA AAA AAA AAA AAG CCT C (19 bp) | SEQ ID NO: 68 |

TABLE 5
Target sequences for C32195818_20 and
C_27292007_10

C32195818_20/RS13419910
TTATTCTACAACATCCCCTTTTATTT[A/G]
ATGATCTGGAAAATTCTGCTTTG SEQ ID NO: 69
C_27292007_10/RS4371338
GATAGGCTGATCTATGTATTTTGAAA[A/G]
CCTGAAAACTTGGCATGTCTTTTCT SEQ ID NO: 70

TABLE 6
Primary samples for SNP analysis

| Diagnosis | Internal ID | Age | Sex |
|---|---|---|---|
| MCL | MCL1 | 49 | M |
| | MCL2 | 77 | F |
| | MCL3 | 78 | M |
| | MCL4 | 77 | F |
| | MCL5 | 73 | M |
| | MCL6 | 44 | F |
| | MCL7 | 70 | M |
| | MCL8 | 79 | M |
| | MCL9 | 58 | F |
| | MCL10 | 68 | F |
| FL | FL1 | 67 | F |
| | FL2 | 61 | F |
| | FL3 | 53 | F |
| | FL4 | 72 | F |
| | FL5 | 49 | F |
| | FL6 | 40 | M |
| | FL7 | 54 | F |
| | FL8 | 72 | F |
| | FL9 | 55 | M |
| | FL10 | 43 | M |
| | FL11 | 56 | F |
| | FL12 | 52 | M |

TABLE 7
Primary samples for epigenetic analysis

| Sample type | Age | Sex | Purification method* | Purity** |
|---|---|---|---|---|
| Tonsil-1 | <5 years | na | | |
| Tonsil-2 | <5 years | na | | |
| Tonsil-3 | <5 years | na | | |
| Tonsil-4 | <5 years | na | CD3 depletion | >95% |
| Tonsil-4 | <5 years | na | CD3 depletion | >95% |
| MCL1 | 57 | M | CD19-coupled Dynabeads | |
| MCL3 | 62 | M | | |
| MCL4 | na | K | | 80% |
| MCL6 | 70 | M | CD19-coupled Dynabeads | 96% |
| FL1 (grade 2) | 56 | F | CD19-coupled Dynabeads | |
| FL2 (grade 1) | 69 | F | CD19-coupled Dynabeads | |
| FL3 (grade 3) | 76 | F | CD19-coupled Dynabeads | |
| FL4 (grade 3) | 85 | M | CD19-coupled Dynabeads | |
| FL5 (grade 3) | 62 | F | CD19-coupled Dynabeads | |
| DLBCL | 44 | M | | |

*All samples purified using Ficoll-Isopaque centrifugation
**measured as CD19 positive, viable cells in flow cytometry
na - information not availble

### REFERENCES

1. Ek S, Dictor M, Jerkeman M, Jirstrom K, Borrebaeck C A. Nuclear expression of the non B-cell lineage Sox11 transcription factor identifies mantle cell lymphoma. Blood. 2008; 111:800-805.
2. Wang X, Asplund A C, Porwit A, et al. The subcellular Sox11 distribution pattern identifies subsets of mantle cell lymphoma: correlation to overall survival. Br J. Haematol. 2008; 143:248-252.
3. Michael Dictor S E, Maria Sundberg, Janina Warenholt, Czabafy György, Sandra Sernbo, Elin Gustaysson, Waleed Abu-Alsoud, Torkel Wadstrom and Carl Borrebaeck. Strong Lymphoid Nuclear Expression of Sox11 Transcription Factor Defines Lymphoblastic Neoplasms, Mantle Cell Lymphoma and Burkitt Lymphoma. Haematologica. 2009.
4. Brennan D J, Ek S, Doyle E, et al. The transcription factor Sox11 is a prognostic factor for improved recurrence-free survival in epithelial ovarian cancer. Eur J. Cancer. 2009; 45:1510-1517.
5. Lee C J, Appleby V J, Orme A T, Chan W I, Scotting P J. Differential expression of SOX4 and SOX11 in medulloblastoma. J. Neurooncol. 2002; 57:201-214.
6. Weigle B, Ebner R, Temme A, et al. Highly specific overexpression of the transcription factor SOX11 in human malignant gliomas. Oncol Rep. 2005; 13:139-144.
7. Brennan D J, Ek S, Doyle E, et al. The transcription factor Sox11 is a prognostic factor for improved recurrence-free survival in epithelial ovarian cancer. Eur J. Cancer. 2009; 45:1510-1517.
8. Azuma T, Ao S, Saito Y, et al. Human SOX11, an upregulated gene during the neural differentiation, has a long 3′ untranslated region. DNA Res. 1999; 6:357-360.
9. Bergsland M, Werme M, Malewicz M, Perlmann T, Muhr J. The establishment of neuronal properties is controlled by Sox4 and Sox11. Genes Dev. 2006; 20:3475-3486.
10. Kuhlbrodt K, Herbarth B, Sock E, Enderich J, Hermans-Borgmeyer I, Wegner M. Cooperative function of POU proteins and SOX proteins in glial cells. J Biol. Chem. 1998; 273:16050-16057.
11. Wegner M. From head to toes: the multiple facets of Sox proteins. Nucleic Acids Res. 1999; 27:1409-1420.
12. Wilson M, Koopman P. Matching SOX: partner proteins and co-factors of the SOX family of transcriptional regulators. Curr Opin Genet Dev. 2002; 12:441-446.
13. Wiebe M S, Nowling T K, Rizzino A. Identification of novel domains within Sox-2 and Sox-11 involved in autoinhibition of DNA binding and partnership specificity. J Biol. Chem. 2003; 278:17901-17911.
14. Andreasson U, Ek S, Merz H, et al. B cell lymphomas express CX3CR1a non-B cell lineage adhesion molecule. Cancer Lett. 2008; 259:138-145.
15. Li L C, Dahiya R. MethPrimer: designing primers for methylation PCRs. Bioinformatics. 2002; 18:1427-1431.
16. Frommer M, McDonald L E, Millar D S, et al. A genomic sequencing protocol that yields a positive display of 5-methylcytosine residues in individual DNA strands. Proc Natl Acad Sci USA. 1992; 89:1827-1831.
17. Bock C, Reither S, Mikeska T, Paulsen M, Walter J, Lengauer T. BiQ Analyzer: visualization and quality control for DNA methylation data from bisulfite sequencing. Bioinformatics, 2005; 21:4067-4068.
18. Rohde C, Zhang Y, Jurkowski T P, Stamerjohanns H, Reinhardt R, Jeltsch A. Bisulfite sequencing Data Presentation and Compilation (BDPC) web server—a useful tool for DNA methylation analysis. Nucleic Acids Res. 2008; 36:e34.
19. Michael Dictor S E, Maria Sundberg, Janina Warenholt, Czabafy György, Sandra Sernbo, Elin Gustaysson, Waleed Abu-Alsoud, Torkel Wadström and Carl Borrebaeck. Strong Lymphoid Nuclear Expression of Sox11 Transcription Factor Defines Lymphoblastic Neoplasms, Mantle Cell Lymphoma and Burkitt Lymphoma. Haematologica; 2009.
20. Ortega-Paino E, Fransson J, Ek S, Borrebaeck C A. Functionally associated targets in mantle cell lymphoma as defined by DNA microarrays and RNA interference. Blood. 2008; 111:1617-1624.
21. Lopez de Silanes I, Quesada M P, Esteller M. Aberrant regulation of messenger RNA 3′-untranslated region in human cancer. Cell Oncol. 2007; 29:1-17.
22. Zheng Y G, Wu J, Chen Z, Goodman M. Chemical regulation of epigenetic modifications: opportunities for new cancer therapy. Med Res Rev. 2008; 28:645-687.
23. Bird A P. CpG-rich islands and the function of DNA methylation. Nature. 1986; 321:209-213.
24. Gardiner-Garden M, Frommer M. CpG islands in vertebrate genomes. J Mol. Biol. 1987; 196:261-282.
25. Herman J G, Baylin S B. Gene silencing in cancer in association with promoter hypermethylation. N Engl J. Med. 2003; 349:2042-2054.
26. Song F, Smith J F, Kimura M T, et al. Association of tissue-specific differentially methylated regions (TDMs) with differential gene expression. Proc Natl Acad Sci USA. 2005; 102:3336-3341.
27. Bird A. DNA methylation patterns and epigenetic memory. Genes Dev. 2002; 16:6-21.
28. Jones P A, Baylin S B. The fundamental role of epigenetic events in cancer. Nat Rev Genet. 2002; 3:415-428.
29. Chim C S, Wong K Y, Loong F, Lam W W, Srivastava G. Frequent epigenetic inactivation of Rb1 in addition to p15 and p16 in mantle cell and follicular lymphoma. Hum Pathol. 2007; 38:1849-1857.
30. Gronbaek K, Ralfkiaer U, Dahl C, et al. Frequent hypermethylation of DBC1 in malignant lymphoproliferative neoplasms. Mod Pathol, 2008; 21:632-638.
31. Hayslip J, Montero A. Tumor suppressor gene methylation in follicular lymphoma: a comprehensive review. Mol. Cancer. 2006; 5:44.
32. Herman J G, Civin C I, Issa J P, Collector M I, Sharkis S J, Baylin S B. Distinct patterns of inactivation of p151NK4B and p161NK4A characterize the major types of hematological malignancies. Cancer Res. 1997; 57:837-841.
33. Reddy J, Shivapurkar N, Takahashi T, et al. Differential methylation of genes that regulate cytokine signaling in lymphoid and hematopoietic tumors. Oncogene. 2005; 24:732-736.
34. Rossi D, Capello D, Gloghini A, et al. Aberrant promoter methylation of multiple genes throughout the clinico-pathologic spectrum of B-cell neoplasia. Haematologica. 2004; 89:154-164.
35. Ushmorov A, Leithauser F, Ritz O, Barth T F, Moller P, Wirth T. ABF-1 is frequently silenced by promoter methylation in follicular lymphoma, diffuse large B-cell lymphoma and Burkitt's lymphoma. Leukemia. 2008; 22:1942-1944.
36. Vishwanatha J K, Salazar E, Gopalakrishnan V K. Absence of annexin I expression in B-cell non-Hodgkin's lymphomas and cell lines. BMC Cancer. 2004; 4:8.
37. Lewin J, Plum A, Hildmann T, et al. Comparative DNA methylation analysis in normal and tumour tissues and in cancer cell lines using differential methylation hybridisation. Int J Biochem Cell Biol. 2007; 39:1539-1550.
38. Ehrich M, Turner J, Gibbs P, et al. Cytosine methylation profiling of cancer cell lines. Proc Natl Acad Sci USA. 2008; 105:4844-4849.
39. Sato N, Fukushima N, Maitra A, et al. Discovery of novel targets for aberrant methylation in pancreatic carcinoma using high-throughput microarrays. Cancer Res. 2003; 63:3735-3742.
40. Qin T, Jelinek J, Si J, Shu J, Issa JP. Mechanisms of resistance to 5-aza-2′-deoxycytidine in human cancer cell lines. Blood. 2009; 113:659-667.
41. Jankowski M P, Cornuet P K, Mcllwrath S, Koerber H R, Albers K M. SRY-box containing gene 11 (Sox11) transcription factor is required for neuron survival and neurite growth. Neuroscience, 2006; 143:501-514.
42. de Bont J M, Kros J M, Passier M M, et al. Differential expression and prognostic significance of SOX genes in pediatric medulloblastoma and ependymoma identified by microarray analysis. Neuro Oncol. 2008; 10:648-660.
43. Liu P, Ramachandran S, Ali Seyed M, et al. Sex-determining region Y box 4 is a transforming oncogene in human prostate cancer cells. Cancer Res. 2006; 66:4011-4019.
44. Pouponnot C, Sii-Felice K, Hmitou I, et al. Cell context reveals a dual role for Maf in oncogenesis. Oncogene. 2006; 25:1299-1310.
45. Yang L, Han Y, Suarez Saiz F, Minden M D. A tumor suppressor and oncogene: the WT1 story. Leukemia. 2007; 21:868-876.
46. Hide T, Takezaki T, Nakatani Y, Nakamura H, Kuratsu J, Kondo T: Sox11 prevents tumorigenesis of glioma-initiating cells by inducing neuronal differentiation. Cancer Res 2009, 69:7953-7959.
47. Fernandez V, Salamero O, Espinet B, Sole F, Royo C, Navarro A, Camacho F, Bea S, Hartmann E, Amador V, et al: Genomic and Gene Expression Profiling Defines Indolent Forms of Mantle Cell Lymphoma. Cancer Res, 70:1408-1418

### EXAMPLE B

In this study, lymphomas were surveyed to determine the range of expression of the mantle cell lymphoma-associated Sox11 transcription factor and its relation to cyclin D1. 172 specimens were immunostained for the Sox11 N and C termini. CCND1 was detected by INC and qRT-PCR; in situ hybridization for t(11;14) was applied where needed.

Nuclear Sox11 was strongly expressed in most B and T-lymphoblastic leukemia/lymphomas, half of childhood Burkitt lymphomas (BL) and only weakly expressed in some hairy cell leukemias. Chronic lymphocytic leukemia/lymphoma, marginal zone and diffuse large B-cell lymphomas were negative for Sox11, as were all cases of intermediate BL/DLBCL, myeloma, Hodgkin and mature T-cell and NK/T-cell lymphomas.

Nuclear Sox11 expression is independent of CCND1 and unlikely to be due to translocations in lymphoid neoplasia. In addition to mantle cell lymphoma, it is strongly expressed in lymphoblastic malignancy and BL.

### Introduction

The Sox11 transcription factor, normally expressed in the developing central nervous system, is aberrantly transcribed and expressed in mantle cell lymphoma (MCL) (1)(2)(3). Common MCL simulators do not express nuclear Sox11 but questions remain as to its relation to cyclin D1 (CCND1). We surveyed most categories of B and T cell lymphomas for Sox11, including plasmacytoma/myeloma (4) and hairy cell leukemia (HCL), which are characterized by elevated CCND1 (5-7).

### Design and Methods

Current WHO clinical, histological and immunophenotypical criteria (8) were used to diagnose 172 previously unreported cases (age range months to 89 years; M:F=1.7:1) on formalin-fixed paraffin sections, with or without ancillary flow cytometric and molecular studies. All biologic material was used according to the research ethics principles established for our institution.

B-cell lymphoma (BCL), T-cell lymphoma (TCL), NK/T-cell lymphoma and Hodgkin lymphoma comprised mature (peripheral) lymphomas and B/T lymphoblastic leukemia/lymphoma comprised the immature category (Table 8). CD5⁺ BCL comprise subgroups within recognized lymphoma entities. Burkitt lymphoma was distinguished by typical starry-sky and nuclear morphology, predominantly intraabominal origin, Ki-67 index >95% and consistent CD10⁺ and BCL2⁻ staining (8). Intermediate Burkitt lymphoma/diffuse large B-cell lymphoma (DL/DLBCL) had a similar proliferation index and starry-sky pattern but were largely nodal and showed nuclear, cellular and immunophenotypic features (strong BCL2⁺ or CD10⁻ in all cases) inconsistent with BL.

### Immunohistochemistry

Sections were, microwaved for antigen retrieval in Tris/EDTA (Sox11 buffer, pH 9, for 8+7 min and then stained on an automatic immunostainer using Sox11 antibodies, as detailed below and as needed a rabbit monoclonal anti-CCND1 antibody (1:70, NeoMarkers, USA). Signal was detected using Envision (Dako) and 3,3″-diaminobenzidine.

### Characterization of Sox11 Antibodies

Two primary rabbit anti-human Sox11 antibodies were raised by the HPR-project (9, 10). The first, Sox11^{N-term}, targets the N-terminus of Sox11 and was used successfully in MCL (2). The immunogen shows some homology with Sox4 but Sox11^{N-term} shows no nuclear reactivity in tonsil sections, known to express Sox4.

Sox11^{C-term}, was raised against the immunogen:

[SEQ ID NO: 79]
EDDDDDDDDDELQLQIKQEPDEEDEEPPHQQLLQPPGQQPSQLLRRYNVA
KVPASPTLSSSAESPEGASLYDEVRAGATSGAGGGSRLYYSFKNITKQHP
PPLAQPALSPASSRSVSTSSS

a 121 aa carboxy terminal peptide, specific for Sox11.

The specificity of both antibodies was verified in the MCL cell lines, SP53 and Granta-519, using a Western blot of extracted proteins, which were separated by reducing SDS-PAGE (NuPAGE 10% Bis-Tris gels, Invitrogen, CA, USA). Each well was loaded with lysate from approximately 6×10⁵ cells and the gel was blotted onto a PVDF membrane (Amersham Hybond-P, GE Healthcare, Sweden) for 30 min (15 V) and blocked overnight in 5% milk/PBS. Sox11^{N-term} or Sox11^{C-term} was applied 1:500 for 30 min. After washing with PBS an HRP-labeled goat anti-rabbit antibody, diluted 1:10,000 was applied. Bands were detected with SuperSignal West Femto Max Sensitivity Substrate (Pierce) according to the manufacturer's protocol.

siRNA Knockdown Study

Washed Granta-519 cells were suspended in 100 μl nucleofector solution (Reactionlab, to Sweden) at 5×10⁶ cells/sample. Each cuvette was then loaded with 50 μmol of siRNA ((Ambion, Austin, USA) consisting of antisense Sox11.1 [pool] UAACGUACCAACAUACUUGuu [SEQ ID NO: 8], UGCGUCACGACAUCUUAUCuu [SEQ ID NO: 9], UCUUCGAGGAGCCUAGAGGuu [SEQ ID NO: 10] and AGACCGACAAGCUUCAAACuu [SEQ ID NO: 11] (or controls using complementary sense oligoRNA), transfected (Amaxa Biosystems, Germany), then incubated in R-10 medium at 37° C. for 3 h, plated at a density of 0.50−0.75×10⁶ cells/ml and grown 2-3 d.

### Quantitative Real-Time PCR

Briefly, reverse transcribed RNA template was used a fluorogenic 5′ nuclease assay to determine C_{T} values on a Rotorgene cycler (Corbett Research). Primers and probes for CCND1 and the reference gene TBP and cycling conditions have been published (11). Each sample was run in triplicate with Granta-519 cDNA as a positive control, one negative water control and two no template controls using DNase I-treated RNA. Gene expressions were calculated to determine the fold increase in normalized CCND1 C_{T} values relative to a benign node calibrator using the appropriate formulae (12).

### Interphase FISH/CISH

We isolated whole nuclei from thick sections digested in 0.5% pepsin. Filtered nuclei were spread on a glass slide, afterfixed in Carnoy's fixative, prehybridized in 0.1% Triton-100, digested in 0.3 mg/mL pronase, rinsed in glycine/PBS, dehydrated in ethanol and air-dried. A dual color, dual fusion translocation probe (Vysis, USA) was hybridized as previously reported (2). Yellow fusion signals are evidence of t(11;14). For each specimen 50 nuclei were scored for the number of fusion signals using the cutoff value 6, which was based on fusion counts in 350 total nuclei from benign nodes and follicular lymphoma.

CISH, chromogenic in situ hybridization, was performed according to the manufacturer's protocol using a mixture of Texas Red- and FITC-labeled probes (Dako DuoCISH™) which target sequences flanking the CCND1 locus. Overlapping blue and red signals indicated co-localization and a split signal indicated a break at the CCND1 locus. Several MCL were used as positive controls.

### Results

Both antibodies yielded a ˜60 kDa band on Western blots corresponding to Sox11 (FIG. 12A); after Sox11 knock-down the band was not detectable using Sox11^{C-term} (FIG. 12B).

Nineteen MCL in the original report were reanalyzed with Sox11^{C-term} and results between the two antibodies were concordant to a high degree aside from occasional differences in staining intensity: one case remained negative with either antibody, one converted to positive (FIG. 12C) and two became immunonegative. Cytoplasmic staining (2) appeared to be reciprocally related to nuclear intensity for both antibodies and was not scored.

Of 23 new MCL specimens, 19 (83%) expressed nuclear Sox11. Five of the 23 were studied with molecular techniques and showed 15 to 99-fold increases in CCND1 expression and between 14 and 72% of nuclei with FISH fusion signals, confirming t(11;14). No consistent relation between CCND1 staining intensity, CCND1 transcription level and the intensity of Sox11 staining was apparent. For example, two MCL showing 22 and 34-fold increases of CCND1 mRNA lacked nuclear Sox11 protein.

Both Sox11 and molecular analysis could differentiate CD5⁺ simulators from MCL (Table 8). Twenty-nine non-MCL, including MZL, CD23⁻ CLL/SLL, CD5⁺ DLBCL and BCL NOS were problematic in their distinction from MCL. Twelve of these were analyzed further and all were negative for t(11;14) by FISH and/or had a normal CCND1 transcription level. All 12 were also immunonegative for nuclear Sox11, whereas all six CCND1⁺ MCL tested with molecular techniques expressed Sox11. As expected, other typical CLUSLL, FL, MZL and DLBCL also lacked Sox11 in the nuclei. Hodgkin lymphoma and T-cell lymphoma subtypes, including NK/T-cell lymphoma, were similarly negative. Most tumors in all categories which lacked nuclear Sox11 produced variably intense cytoplasmic signal, as previously reported (2).

Unexpectedly, we found strong nuclear Sox11 staining in both childhood Burkitt lymphoma (BL) and acute lymphoblastic leukemia/lymphoma, regardless of phenotype (BTT-ALULBL). Seven of fourteen BL were positive and this was reconfirmed with Sox11^{C-term} staining (FIG. 12D). Importantly, none of six high-grade adult B-cell lymphomas intermediate between BL and DLBCL (see footnote in Table 1) was positive with the Sox11^{N-term} antibody (FIG. 12E). Even more strikingly, all ten T-LBL (FIG. 12F) and eight of nine stained B-ALULBL (FIG. 12G) were positive for Sox11^{N-term}. Sox11^{C-term} also confirmed the protein in three B-LBL but was negative in both stained B-ALL; four of five tested T-LBL were also positive with Sox11^{C-term}. Notable was the fact that two T-LBL produced no or weak IHC signal for terminal deoxynucleotidyl transferase (TdT), despite their otherwise typical morphologic and immunophenotypical features. The apparent slight decrease in sensitivity of Sox11^{C-term} compared with Sox11^{N-term} could not be further evaluated due to limited available Sox11^{C-term}.

HCL typically shows modestly elevated CCND1 transcription with weak immunostaining for the protein. Our previous study has shown no upregulation of Sox11 transcription but we nevertheless found very weak Sox11^{N-term} immunostaining in six of 12 (DBA44⁺/Annexin-1⁺) cases (Table 9), which generally paralleled the strength of CCND1 signal, in contrast to the lack of staining covariation noted in MCL. Moreover, in two of three HCL cases tested the presence of Sox11 protein was confirmed with the Sox11^{C-term} antibody but only a single specimen (case 9 in Table 9) produced a moderately strong signal (FIG. 12H-I).

The third subtype with frequent modestly upregulated CCND1 transcription is represented by seven CCND1⁺ myeloma (5)/plasmacytoma (2) and two cases of CCND1⁻ myeloma (Table 8). Regardless of CCND1 status, nuclear Sox11 signal was consistently absent.

### Discussion

The Sox family of transcription factors is widely distributed in animals and Sox proteins are implicated in fundamental developmental processes such as differentiation of murine embryonic stem cells (13), neurogenesis and chondrogenesis (14). Sox11 is expressed in the developing human nervous system (15), medulloblastoma (16) and glioma (17) but has no defined role in B-lymphocyte ontogeny. It is intriguing that the strong nuclear expression of Sox11 in lymphoid neoplasia appears limited to three disparate categories, which include the two mature B-cell tumors, mantle cell lymphoma and true Burkitt lymphoma, and immature lymphoblastic neoplasms.

Interestingly, frequent nuclear Sox11 expression in clinically, morphologically and genetically typical BL was not matched by expression in adult intermediate BUDLBCL.

We reconfirmed nuclear Sox11 expression in the vast majority of prospectively studied MCL. Rare clinically and morphologically typical cases of MCL with or without t(11;14)(q13;q32) may fail to stain for CCND1, using a sensitive rabbit monoclonal antibody (2, 18). This study confirms the consistent Sox11 immunonegativity in the nuclei of common MCL simulators, including the problematic CD5⁺ variants of common peripheral B-cell lymphoma subtypes, for which ancillary molecular techniques may not be available to rule out CCND1⁻ MCL.

The mechanism of Sox11 dysregulation is currently unknown but our negative nuclear Sox11 immunostaining in CCND1⁺ myeloma cells indicates that the protein is not dependent on CCND1. In myeloma, upregulated CCND1 is due to a polysomic chromosome 11 in half of cases, while in about one in six it is due to the same translocation as in MCL, t(11;14)(q13;q32) (4). Moreover, strong Sox11-specific signal occurred at high frequency in Burkitt lymphoma and T and B-lymphoblastic neoplasms, tumors devoid of t(11;14) but which may contain a variety of other translocations, including those involving transcription factors. These facts make it unlikely that any recognized structural or numerical chromosomal changes are a direct cause of elevated Sox11. In contrast, HCL differed markedly from all the above neoplasms in that nuclear Sox11 staining, present in about half of the specimens, was generally very weak and paralleled that of weak or negative cyclin D1, the regulation of which is not due to altered gene dosage or t(11;14) (5). Note that the presence of Sox11 in lymphoblastic leukemia/lymphoma introduces an important caveat in the use of this marker for MCL given that adult lymphoblastic lymphoma is a rare morphologic mimic of MCL.

In conclusion, strong nuclear Sox11 expression in lymphoma is extended to include even lymphoblastic and Burkitt lymphoma, indicating a wider role for the protein in lymphomagenesis than previously reported.

TABLE 8
Lymphoid neoplasia studied for nuclear Sox11

| | | | | Anti- | Anti- | CCND1 | |
|---|---|---|---|---|---|---|---|
| | | | | Sox11^{N-term} | Sox11^{C-term} | mRNA | FISH/CISH for |
| | | | | nuclear | nuclear | (Mean fold | ch.11 |
| B-cell lymphoma | | N | Site | signal | signal | increase) | translocation |
| Mantle cell¹ | CCND1⁺ | 23* | 2 marrow, 1 | 18/23 | ND | 5/5 pos (15-99) | 5/5 pos |
| | | | salivary gland, 1 | pos | | | |
| | | | mucosa, 1 spleen, | | | | |
| | | | 1 chest wall, 17 | | | | |
| | | | node | | | | |
| CLL/SLL² | CD23⁺ | 4 | 3 node, 1 mucosa | 0/4 pos | ND | 0/1 pos | 0/1 pos |
| | CD23⁻ | 3 | 3 node | 0/2 pos | ND | 0/1 pos | 0/3 pos |
| Marginal zone | CD5⁻ | 13 | 4 spleen, 4 node, 1 | 0/13 pos | ND | ND | 0/1 pos |
| | | | thyroid, 2 dermis, 1 | | | | |
| | | | rectum, 1 | | | | |
| | | | conjunctiva | | | | |
| | CD5⁺ | 3 | 1 conjunctiva, 1 | 0/3 pos | ND | 0/1 pos | 0/1 pos |
| | | | breast, 1 orbit | | | | |
| Diffuse large B-cell | CD5⁻ | 26 | 15 node, 3 testis | 0/26 pos | ND | ND | ND |
| | CD5⁺ | 5 | 5 node | 0/5 pos | ND | ND | 0/5 pos |
| Intermediate | BL/DLBCL³ | 6 | 6 node | 0/6 pos | ND | ND | ND |
| Follicular | | 5 | 5 node: 1 gr I, 2 gr | 0/4 pos | ND | ND | ND |
| | | | II, 2 gr III | | | | |
| Myeloma | CCND1⁻ | 2 | 2 marrow | 0/2 pos | ND | ND | ND |
| | CCND1⁺ | 7 | 1 dermis, 1 node, 5 | 0/7 pos | 0/1 pos | ND | ND |
| | | | marrow | | | | |
| Lymphoplas | | 1 | nasopharynx | 0/1 pos | ND | ND | ND |
| macytic | | | | | | | |
| Burkitt⁴ | | 14 | 2 distal ileum, 1 | 7/14 pos | 3/4 pos | ND | ND |
| | | | ovaries/cecum, 7 | | | | |
| | | | abdomen, 2 neck | | | | |
| | | | node, 1 marrow, 1 | | | | |
| | | | tonsil | | | | |
| B-cell, NOS, low | | 1 | 1 node | 0/1 pos | ND | ND | 0/1 pos |
| grade | | | | | | | |

| | | | Anti- | Anti- | |
|---|---|---|---|---|---|
| | | | Sox11^{N-term} | Sox11^{C-term} | |
| | | | nuclear | nuclear | |
| T-cell lymphoma | N | Site | signal | signal | Comments |
| Angioimmunoblastic | 3 | 3 node | 0/3 pos | ND | |
| ALCL, ALK1⁺ | 4 | 4 node | 0/4 pos | ND | |
| nuclear/cytoplasmic | | | | | |
| ALCL, ALK1⁺ cytoplasmic | 1 | 1 node | 0/1 pos | ND | |
| Mycosis fungoides | 1 | 1 skin | 0/1 pos | ND | |
| PTCL, NOS | 4 | 4 node | 0/4 pos | ND | |
| TCL, enteropathy type | 2 | 2 small bowel | 0/2 pos | ND | |
| TCL, hepatosplenic | 1 | 1 spleen | 0/1 pos | ND | TCRα/β⁺ |
| TCL, large granular cell | 1 | 1 spleen | 0/1 pos | ND | |
| T/NK | 4 | 3 nasopharynx, 1 | 0/4 pos | ND | |
| | | nose | | | |
| Lymphoblastic neoplasia | | | | | |
| B-lymphoblastic | 9 | 4 leukemia, 5 | 8/9 pos | 3/5 pos | Age range <1 to 69 years |
| leukemia/lymphoma | | lymphoma | | | |
| T-lymphoblastic lymphoma | 10 | 8 thymus, 2 | 10/10 | 4/5 pos | Age range 1 to 70 years; TdT |
| | | node | pos | | weak in 1 case with strong |
| | | | | | Sox11^{C-term} |
| Hodgkin | | | | | |
| Classic | 5 | 5 node | 0/5 pos | ND | |
| Lymphocyte predominance | 2 | 2 node | 0/2 pos | ND | |

Pos, positive;
ND, not determined
¹Three cases had blastoid morphology.
²Includes a composite CCND1⁺/Sox11⁻ MCL with Sox11⁻ CLL/SLL in same node.
³age range 49 to 82 years (median 76).
⁴age range 5 to 56 years (median 11.5) with all but one still alive (median survival 8 yrs.); two of three cases with t(8; 14) were Sox11⁺

TABLE 9
Hairy cell leukemia expression of CCND1 and Sox11.¹

| Case | Biospy site | CCND1 | Sox11^{N-term} | Sox11^{C-term} |
|---|---|---|---|---|
| 1. | Spleen | − | − | ND |
| 2. | Marrow | (+) | (+) | − |
| 3. | Spleen | − | − | ND |
| 4. | Marrow | (+) | (+) | (+) |
| 5. | Marrow | + | (+) | ND |
| 6. | Marrow | (+) | − | ND |
| 7. | Marrow | + | − | ND |
| 8. | Marrow | (+) | (+) | ND |
| 9. | Marrow | + | (+) | + |
| 10. | Node | (+) | − | ND |
| 11. | Marrow | − | − | ND |
| 12. | Marrow | (+) | (+) | ND |

¹Clinical, morphologic and immunophenotypical (DBA44⁺/annexin-1⁺) HCL
ND, not determined

### EXAMPLE C

### Introduction

Previous survival data has indicated both a pro- and anti-proliferate function of SOX11^{1,2} and emphasise the need of large patient cohorts and experimental data. However, our recent in vitro data indicates a tumour suppressor function for SOX11. Knock-down of SOX11 induces an increase in proliferation in mantle cell lymphoma cell lines (for a list of MCL cell lines used, see example A). Thus, we have used an animal model to demonstrate the in vivo effects of SOX11 silencing.

### Materials and Methods

### Silencing SOX11 in Z138 Mantle Cell Lymphoma Cells

Z138 cells were cultured in RPMI-1640 (HyClone, Sout Logan, Utah) medium supplemented with 10% (v/v) fetal bovine serum (Invitrogen Gibco, Carlsbad, Calif., USA) and 2 mM L-Glutamine (Sigma-Aldrich, St. Louis, Mo., USA), hereafter referred to as R10 medium. shRNA-SOX11 (targeting 5′-CAAGUAUGUUGGUACGUUAuu and 3′-UAACGUACCAACAUACUUGuu) and scrambled control (5′-AGUACUGCUUACGAUACGGUUuu) were introduced into the retroviral vector pRSMX-PG³ using Bgl II and Hind III sites; the vector carries the gene coding for the green fluorescence protein (GFP) as an infection marker. Retroviral particles (with an RD114 envelope) containing the constructs were produced by Vektorenheten (Lund University). The wt Z138 cells were infected overnight with virus at Multiple Of Infection 4, in RPMI-1640, 2 mM L-Glutamine, 8 μg/ml polybrene. As a negative control, wt Z138 cells were treated in the same way, but without the addition of virus. Cells were selected with 5-15 μg/ml of puromycin (InvivoGen, San Diego, USA) until all negative control cells died. The virus-infected, puromycin resistant cells were further analyzed by flow cytometry, and were all positive (100%) for GFP. After removal of puromycin, stable knock-down of SOX11 was achieved in shRNA-SOX11-infected cells compared to the scrambled control, as verified by Q-PCR and WB (data not shown). In Q-PCR experiments, SOX11 gene was amplified using the following primers: 5′-CCCCACAAACCACTCAGACT-3′ and 5′-CCAGGACAGAACCACCTGAT-3′. Western blot was performed using monoclonal anti-SOX11 antibody (Atlas Antibodies, Stockholm, Sweden)

### Animal Care and Injections

NOD-SCID mice were kept at Barriaren, Lund University, Sweden and all procedures were performed with ethical approval (Dnr 229/09) from the local committee (Lund and Malmo djuretiska namnd). 5 or 0.5 million Z138 cells were injected intravenously in the tail of the mice, control mice were injected with PBS. The animal were visually inspected daily and weight twice a week. Animals that showed signs of tumour growth, including abnormal frequency of movement, weight loss or neurological symptoms were sacrificed. All remaining animals were sacrificed after 8 weeks from tumor cell injection, which was the endpoint of the study.

### Results

Animals injected with PBS were monitored for eight weeks without signs of tumor growth. Animals injected with cells with silenced SOX11 or scrambled control cells were sacrificed when (i) signs of tumor growth appeared or (ii) at the end point of 8 weeks after injection. Although both animals from the SOX11-silenced (SOX11^{low}) and scrambled control group (SOX11^{high}) fell sick, our data indicate that animals injected with Z138 cells with silenced SOX11 showed symptoms after a shorter time period, in agreement with previous in vitro data where an increase in the proliferation of lymphoma cells was observed upon SOX11 knock-down (see example A).

### Discussion

SOX11 has recently been shown to be an important diagnostic antigen for MCL.⁴⁻⁷ In this study, a murine model was used to investigate the functional effect of an altered SOX11 level in mantle cell lymphoma cells. Using the mantle cell lymphoma cell line Z138 with altered SOX11 levels, we were able to show that in mice injected with SOX11^{low} the resultant mantle cell lymphoma had a shorter time to symptoms/death related to tumor growth compared to control mice injected with SOX11^{high} tumor cells. Thus, SOX11 is an important target for treatment strategies in mantle cell lymphoma.

### REFERENCES

1. Wang X, Asplund A C, Porwit A, et al. The subcellular Sox11 distribution pattern identifies subsets of mantle cell lymphoma: correlation to overall survival. Br J Haematol. 2008; 143:248-252.
2. Fernandez V, Salamero O, Espinet B, Sole F, Royo C, Navarro A, Camacho F, Bea S, Hartmann E, Amador V, et al: Genomic and Gene Expression Profiling Defines Indolent Forms of Mantle Cell Lymphoma. Cancer Res, 2010; 70:1408-1418
3. Ngo V, Davis e, Lamy L, Yu X, Zhao H, Lenz G, Lam L, Dave S, Yang L, Powell J and Staudt L. A loss-of-function RNA interference screen for molecular targets in cancer. Nature 2006; 441.
4. Ek S, Dictor M, Jerkeman M, Jirstrom K, Borrebaeck C A. Nuclear expression of the non B-cell lineage Sox11 transcription factor identifies mantle cell lymphoma. Blood. 2008; 111:800-805.
5. Wang X, Asplund A C, Porwit A, et al. The subcellular Sox11 distribution pattern identifies subsets of mantle cell lymphoma: correlation to overall survival. Br J. Haematol. 2008; 143:248-252.
6. Michael Dictor S E, Maria Sundberg, Janina Warenholt, Czabafy György, Sandra Sernbo, Elin Gustaysson, Waleed Abu-Alsoud, Torkel Wadström and Carl Borrebaeck. Strong Lymphoid Nuclear Expression of Sox11 Transcription Factor Defines Lymphoblastic Neoplasms, Mantle Cell Lymphoma and Burkitt Lymphoma. Haematologica. 2009.
7. Mozos A, Royo C, Hartmann E, De Jong D, Baro C, Valera A, Fu K, Weisenburger D D, Delabie J, Chuang S S, et al: SOX11 expression is highly specific for mantle cell lymphoma and identifies the cyclin D1-negative subtype. Haematologica 2009, 94:1555-1562.
8. Chen YH, Gao J, Fan G, Peterson L C: Nuclear expression of sox11 is highly associated with mantle cell lymphoma but is independent of t(11;14)(q13;q32) in non-mantle cell B-cell neoplasms. Mod Pathol, 23:105-112.

1. (canceled)

2. A method of treating a cancer in a patient, the method comprising administering to the patient an agent capable of activating Sox11.

3. A method according to claim 2 wherein the cancer is selected from the group consisting of cancers of the breast, bile duct, central nervous system (e.g. brain) and other nerve cells, colon, stomach, reproductive organs, lung and airways, skin, gallbladder, liver, nasopharynx, kidney, prostate, lymph glands, bones (including bone marrow), spleen, blood and gastrointestinal tract.

4. A method according to claim 2 wherein the cancer is a lymphoma or leukaemia.

5. A method according to claim 4 wherein the lymphoma or leukaemia is selected from the group of lymphomas and leukaemias listed in Table 1.

6. A method according to claim 4 wherein the lymphoma or leukaemia is a B cell lymphoma.

7. A method according to claim 5 wherein the cancer is a lymphoma selected from the group consisting of follicular lymphoma (FL), mantle cell lymphoma (MCL) and diffuse large B cell lymphoma (DLBCL).

8.-14. (canceled)

15. A method according to claim 2 wherein the agent is capable of inhibiting the proliferation of cancer cells.

16. A method according to claim 15 wherein the agent is capable of inhibiting the proliferation of cancer cells by 20% or more compared to the proliferation of cancer cells which have not been exposed to the agent, for example by at least 30%, 40%, 50%, 60%, 70%, 80%, 90% or more.

17. A method according to claim 2 wherein the agent is capable of increasing the rate of cancer cell death.

18. A method according to claim 17 wherein the agent is capable of increasing the rate of cancer cell death by 10% or more compared to the rate of cell death of cancer cells which have not been exposed to the agent, for example by at least 20%, 30%, 40%, 50%, 60%, 70%, 80%, 90% or more.

19. A method according to claim 2 wherein the agent increases the transcription, translation, binding properties, biological activity and/or stability of Sox11, and/or signalling induced thereby.

20.-29. (canceled)

30. A method according to claim 2 wherein the agent comprises or consists of a polypeptide according to SEQ ID NO: 1 or a biologically active fragment, variant, fusion or derivative thereof.

31. (canceled)

32. A method according to claim 30 wherein the agent comprises or consists of a biologically active fragment of a polypeptide according to SEQ ID NO: 1.

33. A method according to claim 32 wherein the fragment comprises or consists of at least 100 contiguous amino acid of SEQ ID NO: 1, for example at least 150, 200, 250, 300, 350, 400 or 440 contiguous amino acids of SEQ ID NO: 1.

34. A method according to claim 30 wherein the agent comprises or consists of a biologically active variant of a polypeptide according to SEQ ID NO: 1, or fragment thereof.

35. A method according to claim 34 wherein the variants shares at least 70% sequence identity with a polypeptide according to SEQ ID NO: 1, or fragment thereof, for example at least 80%, 85%, 90%, 95%, 96%, 97%, 98% or 99% sequence identity.

36. A method according to claim 2 wherein the agent comprises or consists of a nucleic acid molecule encoding a polypeptide according to SEQ ID NO: 1 or a biologically active fragment, variant, fusion or derivative thereof.

37.-40. (canceled)

41. A method according to any one of claim 36 wherein the agent comprises or consists of a gene therapy vector.

42.-45. (canceled)

46. A method according to claim 2 wherein the agent comprises a moiety for targeting delivery of the agent to cancer cells.

47.-55. (canceled)

56. A pharmaceutical composition comprising an agent according to claim 2 and a pharmaceutically acceptable excipient, diluent or carrier.

57.-61. (canceled)


SEQUENCE LISTING (80 sequences)

SEQ ID NO: 1 - protein, 441 aa, Homo sapiens
MVQQAESLEAESNLPREALDTEEGEFMACSPVALDESDPDWCKTASGHIKRPMNAFMVWSKIERRKIMEQSPDMHNAEISKRLGKRWKMLKDSEKIPFIREAERLRLKHMADYPDYKYRPRKKPKMDPSAKPSASQSPEKSAAGGGGGSAGGGAGGAKTSKGSSKKCGKLKAPAAAGAKAGAGKAAQSGDYGGAGDDYVLGSLRVSGSGGGGAGKTVKCVFLDEDDDDDDDDDELQLQIKQEPDEEDEEPPHQQLLQPPGQQPSQLLRRYNVAKVPASPTLSSSAESPEGASLYDEVRAGATSGAGGGSRLYYSFKNITKQHPPPLAQPALSPASSRSVSTSSSSSSGSSSGSSGEDADDLMFDLSLNFSQSAHSASEQQLGGGAAAGNLSLSLVDKDLDSFSEGSLGSHFEFPDYCTPELSEMIAGDWLEANFSDLVFTY

SEQ ID NO: 2 - DNA, 8737 bp, Homo sapiens
acctccgcacgagacccagcggcccgggttggagcgtccagccctgcagcggatcatggtgcagcaggcggagagcttggaagcggagagcaacctgccccgggaggcgctggacacggaggagggcgaattcatggcttgcagcccggtggccctggacgagagcgacccagactggtgcaagacggcgtcgggccacatcaagcggccgatgaacgcgttcatggtatggtccaagatcgaacgcaggaagatcatggagcagtctccggacatgcacaacgccgagatctccaagaggctgggcaagcgctggaaaatgctgaaggacagcgagaagatcccgttcatccgggaggcggagcggctgcggctcaagcacatggccgactaccccgactacaagtaccggccccggaaaaagcccaaaatggacccctcggccaagcccagcgccagccagagcccagagaagagcgcggccggcggcggcggcgggagcgcgggcggaggcgcgggcggtgccaagacctccaagggctccagcaagaaatgcggcaagctcaaggcccccgcggccgcgggcgccaaggcgggcgcgggcaaggcggcccagtccggggactacgggggcgcgggcgacgactacgtgctgggcagcctgcgcgtgagcggctcgggcggcggcggcgcgggcaagacggtcaagtgcgtgtttctggatgaggacgacgacgacgacgacgacgacgacgagctgcagctgcagatcaaacaggagccggacgaggaggacgaggaaccaccgcaccagcagctcctgcagccgccggggcagcagccgtcgcagctgctgagacgctacaacgtcgccaaagtgcccgccagccctacgctgagcagctcggcggagtcccccgagggagcgagcctctacgacgaggtgcgggccggcgcgacctcgggcgccgggggcggcagccgcctctactacagcttcaagaacatcaccaagcagcacccgccgccgctcgcgcagcccgcgctgtcgcccgcgtcctcgcgctcggtgtccacctcctcgtccagcagcagcggcagcagcagcggcagcagcggcgaggacgccgacgacctgatgttcgacctgagcttgaatttctctcaaagcgcgcacagcgccagcgagcagcagctggggggcggcgcggcggccgggaacctgtccctgtcgctggtggataaggatttggattcgttcagcgagggcagcctgggctcccacttcgagttccccgactactgcacgccggagctgagcgagatgatcgcgggggactggctggaggcgaacttctccgacctggtgttcacatattgaaaggcgcccgctgctcgctctttctctcggagggtgcagagctgggttccttgggaggaagttgtagtggtgatgatgatgatgataatgatgatgatgatggtggtgttgatggtggcggtggtagggtggaggggagagaagaagatgctgatgatattgataagatgtcgtgacgcaaagaaattggaaaacatgatgaaaattttggtggagttaaagtgaaatgagtagtttttaaacatttttcctgtcctttttttgtcccccctcccttcctttatcgtgtctcaaggtagttgcatacctagtctggagttgtgattattttcccaaaaaatgtgtttttgtaattactatttctttttcctgaaattcgtgattgcaacaaaggcagagggggcggggcgggggaggggaggtaggacccgctccggaaggcgctgtttgaagcttgtcggtctttgaagtctggaagacgtctgcagaggacccttttggcagcacaactgttactctagggagttggtggagatatttttttttcttaagagaacttaaagaactggtgatttttttttaacaaaaaaagggaccattgcaacttttgttaatttaattttttttttttttttttttttttttttttttggagggagaaaactgatgtcttctatgcatccgattcttaacaaaactgcagggagcttgaaaaaatgcagactgtacaaacgcttacaaaaaaaaaaactgtgaactgacttaagatcagagtttacttttcagatcaaattgtttatggttttacaaatgtgatttctacttgccaactttttttttgtaacttgttcccttatacctccttgattgaataccagacagcctagacctcagtacaaaaggtattgaaacatttttgatacataacagacctcagtcttttttaaaaattaatatattttcaggcgtatttttgtacagtgaaaagggaacattcttgctgtgttttttcagtaagactttcaggcacttcttcccttttgatttcttttttttcctctgttttttagcatgcaagtatgttggtacgttatgtcctggtttaaaaaggattaaaattttaaaataatccttgcatctaaaggccttgtggtttaaaaaaaaaaagcaaacttttttttgtacagctatagtagagatttgttcaatatttgtaggtaaagatttattgaaaatggtgatatagacctcagagctgttatcttagtttaaagattgtatatgtactgtactatagtaggactttatgtatctcatacgctgtgatgtggatggggccccagatggaaggtttgaaactggattctcgatttttagcaaaaaagaaaaaaaaaaggcacatagtttaaaaagtttctcattttgtgcaatataatctaaataaagtacagaccatctgcatattttgtagcaaatggtggcaaagcagactcaatgcactgtcgacatcattgcctgtttttttttttttttttttgtgctggaagtctgtatcttgacaattttaataaatcagctggaactgatagaaactcgcatcgccaatagtctctatggaagtcaaactggaggtcctgttgtcgcagagcattcggtggtgaggctgttgtgtgcgcggatgaggggaggtggcaggagagaattctacatttagggggttaggctgaaaagtgttcaattagcaggctgatttctttttcctcttccgctagttgtgaaagacaggggaagggtgttctttctctctgccctccctttccatctccagctccccatttcctttctcacctcctcctccactccctgcctcttctccccacccatcctggcgggcgggctgcgcggaggctcgggagctggccggggaggggcggatggaggggcctgggttgccagctcccttggtcggggtcctgctcgctggggcttgtgtgttctctgcggcgggccgcgtccccgctgagcctcgcggtgacagccgcctttggcagcgagcgctcggggcacttctatccccgcctctcaaagggtggggacagccgtttccagatttgaattttttctgttctttatttttaacgctgcatcttcgcgtgtgctcagaggtggttgttggcggagaacgccgccgcagtgtttgacctctagcggtgaagggggaaggggaagaggaaaggagagaagtggtcggtgtctgtttccttctgtcccccggggccgtggagctgtcggagggaaggaggacggtgcggggccgcagggggcgcggggcgcggcgggacccaggctacgagcgggagggaggcgggagtcgggggaagacgcggcaggccggccgagggcaccccgaggaacatggcatggcctctgtgcgatccgagtcgcggtctccggggtgcctgggagggccgaaccactggtgagggcgtggggagcagggggtggcagagggcacccgggcggtagtccgggacgcgcaaggcagagccctgacgctccgggtccccgtgcctggctcttcttgcctcgccaccgcgtgctcctgggcgcgccccgccgcgggcccttgaggcgcgcggagacaccagcgctggcttcccgggcccgcgggccggggagggaagcctcggggctgcggggtgagaggaagaaagcaaacccggggagcaggcggctgccgcacccgcgcaccccgggccctcaccacgccctccccgcgcgccggctcaggggctgccccggaatcagctccccggggccgccgcaacgaaggtggatccgcatcttgattgttctccgggagcctcctgggggctccggcggcggcgcgggcgcgacccatcccgctggcgctccccgctcgctgaaccccgtttgcctgtccacaccccctcgctccccaccatttttcctgaccggcctgtgtccccgagccctcgcggcaggcccgagcaggcgatcgcggccgggcacgcgcgccccgggctcccgccccccttccgagcatccgccgcctcttttctgctgggtctgggaggagggaggctgggaggccgctcggggcccagcgtgccagccccggagttcagcctcccgagctgcggcgcccgcagcggaggaggttttcagtggctgattgaaactcactgcaaaatcaccacgactctttcacctactgagatgattgaccgaggtttggccttccatttttactgagatttggcgagaccgaatggaagcgtccgcacagtaactgcagctgctaggccagaggggccccggcgcccttcccgcctcccctcccgcttgcttttgccttactcgatcttaccaccacccctcccccggccccccgactgagaactcgggcctctcacccgccccccagcctcccgctctgggcgagcctcctccccagcccccacccctgggatgcgaagccagcaagcttttgctgcagatggacaggtttcttttctgtggctttttcctttcgataaaccatcagatttcagtagtacatttgggaaaagaaggggctgatggcgttaaccaggttctcaatatagaactggatttctggagttgtttaccttaccccacaccccctcaacatgtagactaatgcagccattggtggtacatttattttagccacggataattgaaccagcggtttacaattgacacgtgctccgtgctggtgattttatgtggcagccctctgctgcagttccgaaacttgttggcaacgtaaacccattgataggctgatctatgtattttgaaagcctgaaaacttggcatgtcttttctgttttaatcatagatgaatcttggacattttctgtggtgaggtggaaactttaagtaaattagtaaagtaataatttggcttcagaatgggaagagatagtcaagattttttttttttaaagccatgtggcctaacttgatacaaaaataaaagtaattgtttggcaatctaaatttaaaacctgttagaactcaggacaggcgcttcaatgcgctttttaacaatatttaaggctgttttgatgagtgcgttgtgagaatcatcttaatgaattctttattgagtgtctaaaacatagtataatacacatggtattcttgccactggatagtcttcaataaaagtttaattgatttttttttgttggtctcttaagtaagtcttatttttaactaagcattgacagaatatcttaaaatggtaacctgggggtggcgggtgggtgctgtgtgcacggcagcctagccagtggggatcctgctgtttattataagtagttcacagactctgatggcattttggtaagctttccatctttaagaaattgaaccagcattctcttattaattctttaaactgtggaagtaatttccagttcttacactctgatacgcatcccttttatttaaaaaaaaaaaaaatgctaataaaaggcagtgtacttaaactgtgctttgcaaatattgtgtatgttatgaatgactacagacactgggcaaattatttgtagaatgattatcctttagctagagaaagaaatcattacaactcttttgggcagagatgtttctttttaatgttaatcaaggggaagtgatttaaatatgcataaatgtagcagtcagggtgatttagttgcttttttcatgaaagaaaaagactcaaaagacaagacttatttttctcttctgggacttgaaatcataatcatctgatattagtacagtacaagaaatttacatttgttttttacttcagaatttaagtgacttttgcccaaggaatttgagaaataaggcaaataagttgctctattttaaagtagtcattcaatataaatatattatatcaatcttaacttttttattctctgatatgattaataatatgtatattcttacttttcttctaatgggcatatgtatccttgtggacactttgagagaggttttcttggactctcccatttatagaatctttatactcttttactgtgtggttccctgcttttaacagatttctgaggcaaatatatttgtgcttttttcttatgtaggaagaccagcgaaaatagtttactgagttgtcaattttatcagtagataagaaactttctttattacagtttcagggaagattttttcaggatatttctcagttattctaagggccaaattttgtaaaatttccattaggaatgtcagtttcaaataccctttgtatagcctaagcctgtgaggataacaagaatgagccttacctatcctaacacagggatttacaagttcccaaagtaaccgtctccatgtaactcttgacatacttttctgagatttggcttatttttattattggttatttctcactgttcattctatttgatttattctacaacatccccttttatttaatgatctggaaaattctgctctttgataacaactcaggatttttttgttcagttttggtttttgccccttcctgtggagcctacattttcaaccacaataaagatgaaacaaaatttatgaaactgagctctcttccattttacttactgctggcttttttttttttttttttccttgattcctaccataccttcgtttttttcattgtacttttttaacactacctatatccattagctgcctaattagttttatctgttccatgtggatgcagtgagtttataagagaatttcacaaacaagtagttttttagtgaacttaaaataaacagaattttaaaggagacctatttttatactcaataaaagcacaaaagtgcagaaagtataaaacggcttacaaagggagacacaagctcataatgttccatgtataaaagtaataactttattgggtagagatattcttacaagatctagcacctctgccagtgcacagataggactgttttaaatgatttgggaacttttggttgcctgcagttgtgaacagagaacttctctacagagaaacaaaccactaaaagcaatatgaccgagttgagatgtggtttccaatgagcaattggtgaatttaagcaacctggatgtgcatatgtggaggctcccgtctcactgtttgatcaaacttcttttatgtagtcacgtagacttgattttttctgctgtgaaaatgaaaaaataaagcaatatgacaaaaagtttaaaaatgcataaaaaataggatttcctctaggctcctcgaagagatttttttaatatgatgcttgtcttactttcttagacacgttacatttccccttccaaaaaaaaaaaaaaggacaactggaagtaatttatcatataaagaattttgatcaaatagatattgacaaagggccctctgtcacatttttcttcatccagcttttgttcaaaaacagtatgcctcctcccttgaatcacatagggagaaacgttatactccattctcattaatttcccattttgtctacttttactcttgtacatatgttgtgggtttaagagtcttttgcatttgttctgtgacacctttttttgaattgactgttttaaaacggaggcctattttttccggtttgggactcctagtggttatggcatcccataatgcttcgtgacggccaccaggacagaaccacctgatgttttagagcagttttcagcatgacactgttaacaagtgtgtattttccaaggccacatgaaacttactttcttagccactccaggtttgggagcagaaaagctgaaaaacccttttgtgtagaagtctgagtggtttgtgggggggaccttttttagagtttgcatgccagcgcacggcctattgctgtgaaacagagagaaggtaaagctacctgaggcagtgcgctggaggatgaagtgtttgatagcactaggggggaaagaaaatgcatggcaaagtttcgtcttctcgtagactatctagcatgcagagtgtagtgtgttgaaacggtgtatgacattgctgtatcaaagttgtaaaattaagcattatttattgaaaactatgtatttttttgtaaaaacctgatcacatagagaatatcagtggcttgtgcttgtgcttcgatctaaccagcttcttgacccacccccccttggtatgcagtgttaatgctcagggttgaaaatagtacactccaatgtctcttttgcaagagtttttcacagaggattacatttgttcaaaagactctaataaaattgtgtgatcaatcttcaaaaaaaaaaaaaaaaaaa

SEQ ID NO: 3 - protein, 960 aa, Artificial Sequence
  CDS sequence for the OmicsLink Expression Clone for Sox11
ATGGTGCAGCAGGCGGAGAGCTTGGAAGCGGAGAGCAACCTGCCCCGGGAGGCGCTGGACACGGAGGAGGGCGAATTCATGGCTTGCAGCCCGGTGGCCCTGGACGAGAGCGACCCAGACTGGTGCAAGACGGCGTCGGGCCACATCAAGCGGCCGATGAACGCGTTCATGGTATGGTCCAAGATCGAACGCAGGAAGATCATGGAGCAGTCTCCGGACATGCACAACGCCGAGATCTCCAAGAGGCTGGGCAAGCGCTGGAAAATGCTGAAGGACAGCGAGAAGATCCCGTTCATCCGGGAGGCGGAGCGGCTGCGGCTCAAGCACATGGCCGACTACCCCGACTACAAGTACCGGCCCCGGAAAAAGCCCAAAATGGACCCCTCGGCCAAGCCCAGCGCCAGCCAGAGCCCAGAGAAGAGCGCGGCCGGCGGCGGCGGCGGGAGCGCGGGCGGAGGCGCGGGCGGTGCCAAGACCTCCAAGGGCTCCAGCAAGAAATGCGGCAAGCTCAAGGCCCCCGCGGCCGCGGGCGCCAAGGCGGGCGCGGGCAAGGCGGCCCAGTCCGGGGACTACGGGGGCGCGGGCGACGACTACGTGCTGGGCAGCCTGCGCGTGAGCGGCTCGGGCGGCGGCGGCGCGGGCAAGACGGTCAAGTGCGTGTTTCTGGATGAGGACGACGACGACGACGACGACGACGACGAGCTGCAGCTGCAGATCAAACAGGAGCCGGACGAGGAGGACGAGGAACCACCGCACCAGCAGCTCCTGCAGCCGCCGGGGCAGCAGCCGTCGCAGCTGCTGAGACGCTACAACGTCGCCAAAGTGCCCGCCAGCCCTACGCTGAGCAGCTCGGCGGAGTCCCCCGAGGGAGCGAGCCTCTACGACGAGGTGCGGGCCGGCGCGACCTCGGGCGCCGGGGGCGGCAGCCGCCTCTACTACAGCTTCAAGAACATCACCAAG

SEQ ID NO: 4 - RNA, 21 nt, Artificial Sequence
  siRNA
caaguauguugguacguuauu

SEQ ID NO: 5 - RNA, 21 nt, Artificial Sequence
  siRNA
gauaagaugucgugacgcauu

SEQ ID NO: 6 - RNA, 21 nt, Artificial Sequence
  siRNA
ccucuaggcuccucgaagauu

SEQ ID NO: 7 - RNA, 21 nt, Artificial Sequence
  siRNA
guuugaagcuugucggucuuu

SEQ ID NO: 8 - RNA, 21 nt, Artificial Sequence
  siRNA
uaacguaccaacauacuuguu

SEQ ID NO: 9 - RNA, 21 nt, Artificial Sequence
  siRNA
ugcgucacgacaucuuaucuu

SEQ ID NO: 10 - RNA, 21 nt, Artificial Sequence
  siRNA
ucuucgaggagccuagagguu

SEQ ID NO: 11 - RNA, 21 nt, Artificial Sequence
  siRNA
agaccgacaagcuucaaacuu

SEQ ID NO: 12 - DNA, 20 bp, Artificial Sequence
  PCR primer sox11-1f
aaagcggggtgccgaggact

SEQ ID NO: 13 - DNA, 22 bp, Artificial Sequence
  PCR primer sox11-1r
cttgagcttgccgcatttcttg

SEQ ID NO: 14 - DNA, 21 bp, Artificial Sequence
  PCR primer sox11-2f
agccagagcccagagaagagc

SEQ ID NO: 15 - DNA, 20 bp, Artificial Sequence
  PCR primer sox11-2r
ctgctggacgaggaggtgga

SEQ ID NO: 16 - DNA, 24 bp, Artificial Sequence
  PCR primer sox11-3f
gccgcctctactacagcttcaaga

SEQ ID NO: 17 - DNA, 24 bp, Artificial Sequence
  PCR primer sox11-3r
caatttctttgcgtcacgacatct

SEQ ID NO: 18 - DNA, 24 bp, Artificial Sequence
  PCR primer sox11-4f
ccttgggaggaagttgtagtggtg

SEQ ID NO: 19 - DNA, 28 bp, Artificial Sequence
  PCR primer sox11-4r
cacatttgtaaaaccataaacaatttga

SEQ ID NO: 20 - DNA, 24 bp, Artificial Sequence
  PCR primer sox11-5f
ttggagggagaaaactgatgtctt

SEQ ID NO: 21 - DNA, 24 bp, Artificial Sequence
  PCR primer sox11-5r
ccatccacatcacagcgtatgaga

SEQ ID NO: 22 - DNA, 27 bp, Artificial Sequence
  PCR primer sox11-6f
tgaaaatggtgatatagacctcagagc

SEQ ID NO: 23 - DNA, 24 bp, Artificial Sequence
  PCR primer sox11-6r
aagaacacccttcccctgtctttc

SEQ ID NO: 24 - DNA, 24 bp, Artificial Sequence
  PCR primer sox11-7f
tttagggggttaggctgaaaagtg

SEQ ID NO: 25 - DNA, 24 bp, Artificial Sequence
  PCR primer sox11-7r
aaggaaacagacaccgaccacttc

SEQ ID NO: 26 - DNA, 22 bp, Artificial Sequence
  PCR primer sox11-8f
cgtgtgctcagaggtggttgtt

SEQ ID NO: 27 - DNA, 22 bp, Artificial Sequence
  PCR primer sox11-8r
tcccggagaacaatcaagatgc

SEQ ID NO: 28 - DNA, 23 bp, Artificial Sequence
  PCR primer sox11-9f
ctgcggggtgagaggaagaaagc

SEQ ID NO: 29 - DNA, 24 bp, Artificial Sequence
  PCR primer sox11-9r
gggtggtggtaagatcgagtaagg

SEQ ID NO: 30 - DNA, 24 bp, Artificial Sequence
  PCR primer sox11-10f
ggtttggccttccatttttactga

SEQ ID NO: 31 - DNA, 24 bp, Artificial Sequence
  PCR primer sox11-10r
cctcaccacagaaaatgtccaaga

SEQ ID NO: 32 - DNA, 24 bp, Artificial Sequence
  PCR primer sox11-11f
ttggcaacgtaaacccattgatag

SEQ ID NO: 33 - DNA, 24 bp, Artificial Sequence
  PCR primer sox11-11r
gcttaccaaaatgccatcagagtc

SEQ ID NO: 34 - DNA, 24 bp, Artificial Sequence
  PCR primer sox11-12f
acacatggtattcttgccactgga

SEQ ID NO: 35 - DNA, 24 bp, Artificial Sequence
  PCR primer sox11-12r
tctcaaattccttgggcaaaagtc

SEQ ID NO: 36 - DNA, 24 bp, Artificial Sequence
  PCR primer sox11-13f
ttctcttctgggacttgaaatcat

SEQ ID NO: 37 - DNA, 24 bp, Artificial Sequence
  PCR primer sox11-13r
catggagacggttactttgggaac

SEQ ID NO: 38 - DNA, 24 bp, Artificial Sequence
  PCR primer sox11-14f
ccctttgtatagcctaagcctgtg

SEQ ID NO: 39 - DNA, 22 bp, Artificial Sequence
  PCR primer sox11-14r
tgcactggcagaggtgctagat

SEQ ID NO: 40 - DNA, 24 bp, Artificial Sequence
  PCR primer sox11-15f
cggcttacaaagggagacacaagc

SEQ ID NO: 41 - DNA, 24 bp, Artificial Sequence
  PCR primer sox11-15r
atgtgattcaagggaggaggcata

SEQ ID NO: 42 - DNA, 24 bp, Artificial Sequence
  PCR primer sox11-16f
cacgttacatttccccttccaaaa

SEQ ID NO: 43 - DNA, 24 bp, Artificial Sequence
  PCR primer sox11-16r
gctatcaaacacttcatcctccag

SEQ ID NO: 44 - DNA, 26 bp, Artificial Sequence
  PCR primer sox11-17f
tgtgtagaagtctgagtggtttgtgg

SEQ ID NO: 45 - DNA, 24 bp, Artificial Sequence
  PCR primer sox11-17r
atcttcaagcctgtccctgacatc

SEQ ID NO: 46 - DNA, 18 bp, Artificial Sequence
  PCR primer sox11-f1b
aacttgcccaggaaggtg

SEQ ID NO: 47 - DNA, 18 bp, Artificial Sequence
  PCR primer sox11_f2b
gtgccaagacctccaagg

SEQ ID NO: 48 - DNA, 18 bp, Artificial Sequence
  PCR primer sox11_r2b
tgctgcttggtgatgttc

SEQ ID NO: 49 - DNA, 17 bp, Artificial Sequence
  PCR primer f10n1a
agcgtccgcacagtaac

SEQ ID NO: 50 - DNA, 18 bp, Artificial Sequence
  PCR primer f10n1b
cccttcttttcccaaatg

SEQ ID NO: 51 - DNA, 17 bp, Artificial Sequence
  PCR primer f10n2a
gatgcgaagccagcaag

SEQ ID NO: 52 - DNA, 20 bp, Artificial Sequence
  PCR primer f10n2b
acctcaccacagaaaatgtc

SEQ ID NO: 53 - DNA, 20 bp, Artificial Sequence
  PCR primer f12n1a
tctgatggcattttggtaag

SEQ ID NO: 54 - DNA, 23 bp, Artificial Sequence
  PCR primer f12n2a
aaaaaaaaaaatgctaataaaag

SEQ ID NO: 55 - DNA, 22 bp, Artificial Sequence
  PCR primer f12n3a
ttttttttaaataaaagggatg

SEQ ID NO: 56 - DNA, 22 bp, Artificial Sequence
  PCR primer 16fb
cccttccaaaaaaaaaaaaaag

SEQ ID NO: 57 - DNA, 22 bp, Artificial Sequence
  PCR primer 15rb
gttgtccaaaaaaaaaaaaaac

SEQ ID NO: 58 - DNA, 20 bp, Artificial Sequence
  PCR primer 6if
gcaaaaaagaaaaaaaaaag

SEQ ID NO: 59 - DNA, 21 bp, Artificial Sequence
  PCR primer 6ir
ccttttttttttcttttttgc

SEQ ID NO: 60 - DNA, 20 bp, Artificial Sequence
  PCR primer sox11_r10b
cttcccattctgaagccaaa

SEQ ID NO: 61 - DNA, 16 bp, Artificial Sequence
  PCR primer f4b
ttttttttttggaggg

SEQ ID NO: 62 - DNA, 17 bp, Artificial Sequence
  PCR primer r4b
ttttttttttgtaagcg

SEQ ID NO: 63 - DNA, 21 bp, Artificial Sequence
  PCR primer f5i1
gttggtttaaaaaaaaaaagc

SEQ ID NO: 64 - DNA, 27 bp, Artificial Sequence
  PCR primer f6i2
gcctgtttttttttttttttttttgtg

SEQ ID NO: 65 - DNA, 24 bp, Artificial Sequence
  PCR primer f11i1
gtcaagattttttttttttaaagc

SEQ ID NO: 66 - DNA, 20 bp, Artificial Sequence
  PCR primer f15i1
gtccttttttttttttttgg

SEQ ID NO: 67 - DNA, 19 bp, Artificial Sequence
  PCR primer f14i1
ttttttttttttttccttg

SEQ ID NO: 68 - DNA, 19 bp, Artificial Sequence
  PCR primer r14i1
aaaaaaaaaaaaaagcctc

SEQ ID NO: 69 - DNA, 52 bp, Artificial Sequence
  Target sequence for C32195818_20 /RS13419910
ttattctacaacatccccttttatttdatgatctggaaaattctgctctttg

SEQ ID NO: 70 - DNA, 52 bp, Artificial Sequence
  Target sequence for C_27292007_10/RS4371338
gataggctgatctatgtattttgaaadcctgaaaacttggcatgtcttttct

SEQ ID NO: 71 - DNA, 20 bp, Artificial Sequence
  PCR primer
ccaggacagaaccacctgat

SEQ ID NO: 72 - DNA, 20 bp, Artificial Sequence
  PCR primer
ccccacaaaccactcagact

SEQ ID NO: 73 - DNA, 19 bp, Artificial Sequence
  PCR primer
tggtatcgtggaaggactc

SEQ ID NO: 74 - DNA, 19 bp, Artificial Sequence
  PCR primer
agtagaggcagggatgatg

SEQ ID NO: 75 - DNA, 22 bp, Artificial Sequence
  PCR primer
ggtggataaggatttggattcg

SEQ ID NO: 76 - DNA, 18 bp, Artificial Sequence
  PCR primer
gctccggcgtgcagtagt

SEQ ID NO: 77 - DNA, 20 bp, Artificial Sequence
  PCR primer
gtttggccatacgcaaagat

SEQ ID NO: 78 - DNA, 20 bp, Artificial Sequence
  PCR primer
gaggattggctgacaagagc

SEQ ID NO: 79 - protein, 121 aa, Artificial Sequence
  Sox11 immunogen
EDDDDDDDDDELQLQIKQEPDEEDEEPPHQQLLQPPGQQPSQLLRRYNVAKVPASPTLSSSAESPEGASLYDEVRAGATSGAGGGSRLYYSFKNITKQHPPPLAQPALSPASSRSVSTSSS

SEQ ID NO: 80 - RNA, 23 nt, Artificial Sequence
  siRNA
aguacugcuuacgauacgguuuu